(19) United States
(12) Patent Application Publication
(10) Pub. No.: US 2025/0302545 A1
(43) Pub. Date: Oct. 2, 2025

(54) UPDATING INSTRUMENT NAVIGATION

(71) Applicant: Auris Health, Inc., Santa Clara, CA (US)

(72) Inventors: Daniel Lau, Oakland, CA (US); Jeffrey Adam Kasten, San Mateo, CA (US); Mohammad Matinfar, San Jose, CA (US); Namita Anil Kumar, Redwood City, CA (US); Daniel Shalom Eliahu, Alamo, CA (US); Mali Shen, Sunnyvale, CA (US); Hedyeh Rafii-Tari, Mountain View, CA (US); Qasim Mahmood, Camarillo, CA (US); Chauncey F. Graetzel, Palo Alto, CA (US); Gang Dong, Redwood City, CA (US); Melinda Joy Cromie Lear, San Jose, CA (US); Sarah Catherine Denning, Mountain View, CA (US); Bridget Branton, Redwood City, CA (US); Stacie Marie Niese, San Mateo, CA (US); Roshanak Iranitalab, Redwood City, CA (US)

(73) Assignee: Auris Health, Inc., Santa Clara, CA (US)

(21) Appl. No.: 19/090,297

(22) Filed: Mar. 25, 2025

Related U.S. Application Data
(60) Provisional application No. 63/572,135, filed on Mar. 29, 2024.

(51) Int. Cl. A61B 34/20; A61B 6/03; A61B 6/40; A61B 6/46

(52) U.S. Cl. CPC A61B 34/20; A61B 6/032; A61B 6/4085; A61B 6/463; A61B 6/469; A61B 2034/2051

(57) ABSTRACT

Techniques for updating navigation information associated with an instrument are discussed herein. For example, graphical interface data can be provided during a procedure to assist a user navigating the instrument within the anatomy to reach a target and/or other locations within the anatomy. The graphical interface data can indicate a first spatial relationship between the instrument and the target. Further, image data from an externally located imaging system can be used to determine a second spatial relationship between the instrument and the target. The graphical interface data can be updated based on the second spatial relationship.

### CROSS-REFERENCE TO RELATED APPLICATIONS

This application claims priority and benefit under 35 U.S.C. § 119(e) to U.S. Provisional Patent Application No. 63/572,135, filed Mar. 29, 2024, which is incorporated herein by reference in its entirety.

### TECHNICAL FIELD

This disclosure relates generally to medical systems, and specifically to techniques for updating instrument navigation.

### BACKGROUND

The present disclosure relates to the field of medical procedures. In examples, a medical procedure involves navigating an instrument within anatomy to reach a treatment site. Certain navigation information can be provided to assist a user in navigating the instrument. However, in some cases, the navigation information can inaccurately depict the location of the instrument within the anatomy.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

The present disclosure relates to systems, devices, and methods to determine and/or update a location of an instrument during a procedure. For example, navigation information can be provided during a procedure to assist a user navigating the instrument within the anatomy to reach a target and/or other locations within the anatomy. The navigation information can indicate a spatial relationship between the instrument and the target. To maintain/provide accurate navigation information, image data from an imaging system located outside of the anatomy can be used to determine a spatial relationship between the instrument and the target. The navigation information can be updated based on the spatial relationship determined from the image data.

One innovative aspect of the subject matter of this disclosure can be implemented in a medical system including an imaging system and control circuitry. The control circuitry is configured to generate a graphical interface depicting a first spatial relationship between an instrument and a target within the anatomy; receive image data depicting the anatomy having the instrument disposed therein, where the image data is captured by the imaging system while positioned external to the anatomy; determine a second spatial relationship between the instrument and the target within the anatomy based at least in part on the image data; and update the graphical interface to depict the second spatial relationship between the instrument and the target.

Another innovative aspect of the subject matter of this disclosure can be implemented in a method performed by a controller for a medical system. The method includes steps of generating a graphical interface depicting a first spatial relationship between an instrument and a target within an anatomy; receiving image data depicting the anatomy having the instrument disposed therein, where the image data is captured by an imaging system positioned external to the anatomy; determining a second spatial relationship between the instrument and the target within the anatomy based at least in part on the image data; and updating the graphical interface to depict the second spatial relationship between the instrument and the target.

Another innovative aspect of the subject matter of this disclosure can be implemented in a controller for a medical system, including a processing system and a memory. The memory stores instructions that, when executed by the processing system, cause the controller to generate a graphical interface depicting a first spatial relationship between an instrument and a target within an anatomy; receive image data depicting the anatomy having the instrument disposed therein, where the image data is captured by an imaging system positioned external to the anatomy; determine a second spatial relationship between the instrument and the target within the anatomy based at least in part on the image data; and update the graphical interface to depict the second spatial relationship between the instrument and the target.

Although certain aspects, advantages, and/or features have been described, it is to be understood that not necessarily all such advantages may be achieved in accordance with any particular example. Thus, the disclosed features can be carried out in a manner that achieves or optimizes one advantage or group of advantages as taught herein without necessarily achieving other advantages as may be taught or suggested herein.

### BRIEF DESCRIPTION OF THE DRAWINGS

The disclosed aspects will hereinafter be described in conjunction with the appended drawings, provided to illustrate and not to limit the disclosed aspects, wherein like designations denote like elements.

FIG. 1 illustrates a medical system in accordance with one or more examples.

FIG. 2 illustrates example components of the control system and robotic system of FIG. 1 in accordance with one or more examples.

FIG. 3 is a block diagram illustrating a localization system in accordance with one or more examples.

FIGS. 4A and 4B illustrate an example flow diagram of a process for updating navigation guidance in accordance with one or more examples.

FIG. 5 illustrates an example flow diagram of a process for determining a spatial relationship between elements depicted within image data in accordance with one or more examples.

FIG. 6 illustrates an example flow diagram of a process for providing navigation guidance for a medical instrument in accordance with one or more examples.

FIG. 7 illustrates an example interface to provide intraoperative data and/or receive input regarding a location of a target in accordance with one or more examples.

FIG. 8 illustrates an example interface to provide intraoperative data and/or receive input regarding a location of an instrument in accordance with one or more examples.

FIG. 9 illustrates an example interface to provide information and/or receive input regarding an alignment of an instrument indicator on image data in accordance with one or more examples.

FIG. 10-1 illustrates an example diagram of elements associated with initial navigation information in accordance with one or more examples.

FIG. 10-2 illustrates an example diagram of elements associated with updated navigation information in accordance with one or more examples.

FIG. 11-1 illustrates an example interface to provide graphical interface data associated with initial navigation information in accordance with one or more examples.

FIG. 11-2 illustrates an example interface to provide graphical interface data associated with updated navigation information in accordance with one or more examples.

FIG. 12 shows a block diagram of an example controller for a medical system, according to some implementations.

FIG. 13 shows an illustrative flowchart depicting an example operation for guiding navigation of an instrument within an anatomy, according to some implementations.

### DETAILED DESCRIPTION

Certain medical procedures are associated with a preoperative phase where an anatomical map is generated and a target is designated and an intraoperative phase where the target is found. For example, during a preoperative phase, an anatomical map can be created and a location within the anatomy can be designated as a target. During an intraoperative phase, a scope can be controlled in an attempt to reach the target. However, in some situations, differences in the characteristics of the anatomy between preoperative and intraoperative phases, distortion introduced into an environment and sensed by a sensor, and/or other factors, can make it difficult to accurately navigate to the same preoperative target. For instance, in the context of a bronchoscopy, the lungs can experience different levels of inspiration, deform due to interaction with an instrument, and/or shift/move between preoperative and intraoperative phases. Further, a system used to detect a location of the instrument can experience distortion or other errors/problems and/or a patient can be positioned differently between preoperative and intraoperative phases. Such issues can lead to variations of the anatomical map between preoperative and intraoperative phases, potentially causing inaccuracies in determining the location of the scope.

The present disclosure relates to systems, devices, and methods to localize and/or update a location of a medical instrument during a procedure, such as a position of the medical instrument relative to a target and/or anatomy. For example, a medical system can provide navigation information during a procedure to assist a user navigating a medical instrument within the anatomy to reach a target and/or other locations within the anatomy. In some cases, the navigation information can include a map of the anatomy, a location of the medical instrument, and/or a location of the target, wherein one or more pieces of the navigation information can be based on preoperative data. For example, the navigation information can provide a map and/or location of the target that is based on preoperative data and provide a location of the medical instrument obtained from real-time/intraoperative sensor data and/or image data.

During the procedure (e.g., the intraoperative phase), image data from an externally located imaging system can be used to accurately determine the location of the medical instrument and update the navigation information, if needed. For example, the imaging system can be positioned over the anatomy and configured to capture an internal image of the anatomy from an external position, such as a Computed Tomography (CT) system positioned in proximity to a patient. The medical system can receive the image data and analyze the image data to determine a spatial relationship between the medical instrument and the target. In examples, the medical system can display the image data to a user, receive user input indicating a position of the medical instrument and/or a position of the target in the image data, and analyze the image data based on the user input and/or image processing techniques (e.g., including models associated with machine learning/artificial intelligence) to determine a spatial relationship between the medical instrument and the target. The medical system can update the navigation information based on the spatial relationship indicated/identified in the image data to reflect the current/intraoperative position of the medical instrument relative to the target and/or the map. Thereby, the navigation information can accurately depict the position of the medical instrument, position of the target, and/or other information regarding the anatomy.

Although examples are discussed in the context of a medical procedure with multiple phases, the techniques can be used during the same phase of a procedure, such as to accurately identify a current location of a medical instrument relative to a target, wherein the target was previously identified in the same phase of a procedure.

In examples, the techniques discussed herein use real-time/intraoperative image data from an imaging system located externally to an anatomical site. In some cases, the imaging system provides 3D image data that enables a user to accurately identify a location of an instrument and/or a location of a target intraoperatively. This can allow navigation guidance to be updated, allow the user to view or confirm that the instrument is located appropriately relative to a target/anatomy (which can be advantageous in procedures that require a relatively high level of precision), etc. In examples, using 3D image data can be more advantageous than 2D image data. For instance, in some situations, certain targets/anatomical feature are not visible in 2D image data, but are visible in 3D image data. By providing 3D image data, certain targets/anatomical features can be identified, more easily identified, or more accurately identified (e.g., in multiple slices of image data). However, the techniques can be implemented with 2D image data in many cases and still provide various advantages. In any event, image data from an external imaging system can be used to update navigation guidance, view a location of an instrument relative to a target/anatomy, or otherwise enhance navigation or other functions.

In some cases, the techniques discussed herein implement robotic-assisted medical procedures, wherein robotic tools/components enable a physician, operator, or other user to perform procedures. For example, the robotic tools can engage with and/or control one or more medical instruments, such as a scope, to access an anatomical site in a patient and/or perform a diagnosis or treatment at the anatomical site. In some cases, the robotic tools are guided/controlled by a physician, operator, or other user. In other cases, the robotic tools operate in an automatic or semi-automatic manner. Although many techniques are discussed in the context of robotic-assisted medical procedures, the techniques can be applicable to other types of medical procedures, such as procedures that do not implement robotic tools or implement robotic tools for relatively few operations (e.g., less than a threshold number). For example, the techniques can be applicable to procedures in which a manually operated medical instrument is implemented, such as a manual scope controlled entirely by a physician, operator, or other user. In examples discussed herein, the techniques/systems are implemented in the context of diagnosis, therapy/treatment, etc.

Although certain examples are disclosed herein, the subject matter extends beyond the specifically disclosed examples to other alternative examples and/or uses, and to modifications and/or equivalents thereof. Thus, the scope of the claims appended hereto is not limited by any of the particular examples described herein. For example, in any method or process disclosed herein, the acts or operations of the method or process can be performed in any suitable sequence and are not necessarily limited to any particular disclosed sequence. Various operations can be described as multiple discrete operations in turn, in a manner that may be helpful in understanding certain examples; however, the order of description should not be construed to imply that these operations are order dependent. Additionally, the structures, systems, and/or devices described herein can be embodied as integrated components or as separate components.

Certain aspects and advantages of these examples are described. Not necessarily all such aspects or advantages are achieved by any particular example. Thus, for example, various examples can be carried out in a manner that achieves or optimizes one advantage or group of advantages as taught herein without necessarily achieving other aspects or advantages as may also be taught or suggested herein.

### Example Medical System

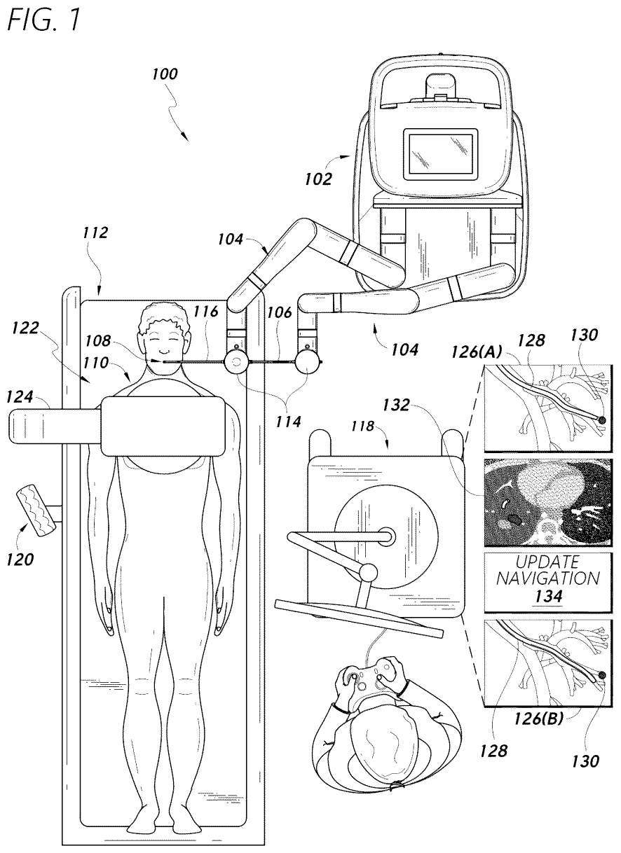

FIG. 1 illustrates an example medical system 100 (also referred to as “surgical medical system 100” or “robotic medical system 100”) in accordance with one or more examples. For example, the medical system 100 can be arranged for diagnostic and/or therapeutic bronchoscopy, as shown. The medical system 100 can include and utilize a robotic system 102, which can be implemented as a robotic cart, for example. Although the medical system 100 is shown as including various cart-based systems/devices, the concepts disclosed herein can be implemented in any type of robotic system/arrangement, such as robotic systems employing rail-based components, table-based robotic end-effectors/manipulators, etc. The robotic system 102 can comprise one or more robotic arms 104 (also referred to as “robotic positioner(s) 104”) configured to position or otherwise manipulate a medical instrument, such as a medical instrument 106 (e.g., a steerable endoscope or another elongate instrument). For example, the medical instrument 106 can be advanced through a natural orifice access point (e.g., the mouth 108 of a patient 110, positioned on a table 112 in the present example) to deliver diagnostic and/or therapeutic treatment. Although described in the context of a bronchoscopy procedure, the medical system 100 can be implemented for other types of procedures, such as gastro-intestinal (GI) procedures, renal/urological/nephrological procedures, etc.

With the robotic system 102 properly positioned, the medical instrument 106 can be inserted into the patient 110 robotically, manually, or a combination thereof. In examples, the one or more robotic arms 104 and/or instrument driver(s) 114 thereof can control the medical instrument 106. In some implementations, the medical instrument 106 can be advanced within a sheath 116, which can be coupled to, and/or controlled by, a robotic arm in some cases. For example, the medical instrument 106 and the sheath 116 can each be coupled to a separate instrument driver from a set of instrument driver(s) 114. The instrument driver(s) 114 can be repositionable in space by manipulating the one or more robotic arms 104 into different angles and/or positions.

In the example shown, the medical instrument 106 can be directed down the patient's trachea and lungs after insertion and/or advanced to a target destination or operative site. In examples, in order to enhance navigation through the patient's lung network and/or reach the desired target, the medical instrument 106 can be manipulated to telescopically extend from the outer sheath 116 to obtain enhanced articulation and/or greater bend radius. The use of separate instrument driver(s) 114 can allow the medical instrument 106 and sheath 116 to be driven independently of each other.

In examples, the medical instrument 106 includes an elongate member/shaft configured to be inserted/retracted, articulated, or otherwise moved within the anatomy. Further, in examples, the medical instrument 106 includes an imaging device(s) (e.g., camera) positioned on a distal end of the elongate shaft and/or deployed through a working channel of the elongate shaft. The imaging device(s) can be configured to generate/capture image data and/or send the image data to another device/component. Moreover, in examples, the medical instrument 106 includes an instrument base/handle(s) positioned at a proximal end of the medical instrument 106. The instrument base(s) can be configured to couple to a manipulator (e.g., end of a robotic arm). The instrument base can include a drive input(s) configured to couple to a drive output(s) of the manipulator, wherein the drive input(s) and/or drive output(s) act as an interface.

Moreover, in examples, the medical instrument 106 includes a working channel(s) configured to receive one or more other instruments/elements therein and/or provide other functionality. The working channel(s) can extend axially, such as along the length of the medical instrument 106. Furthermore, the medical instrument 106 can include or be associated with an elongate movement member(s) (e.g., pulls wires) that can extend from a proximal end through the elongate shaft to the distal end of the elongate shaft. The elongate movement member(s) can be manipulated, such as by manipulators on the one or more robotic arms 104, to control actuation of the elongate movement member(s).

Furthermore, in examples, the medical instrument 106 includes a sensor(s), such as an electromagnetic (EM) sensor(s), shape sensor(s) (e.g., shape sensing fiber), accelerometer(s), gyroscope(s), satellite-based positioning sensor(s) (e.g., global positioning system (GPS) sensors), radio-frequency transceiver(s), and so on. The sensor(s) (also referred to as “position sensor(s)”) can be configured to generate sensor data and/or provide the sensor data to another device/component. The sensor(s) can be disposed at a distal end of the elongate shaft, along a length of the elongate shaft, etc. Further, in examples, the medical instrument 106 is configured to receive an elongate member/device through a working channel, wherein the elongate member includes one or more sensors along a length of the elongate member. In some examples, a sensor on the medical instrument 106 can provide sensor data to control circuitry of the medical system 100, which is then used to determine a position, orientation, and/or shape of the medical instrument 106.

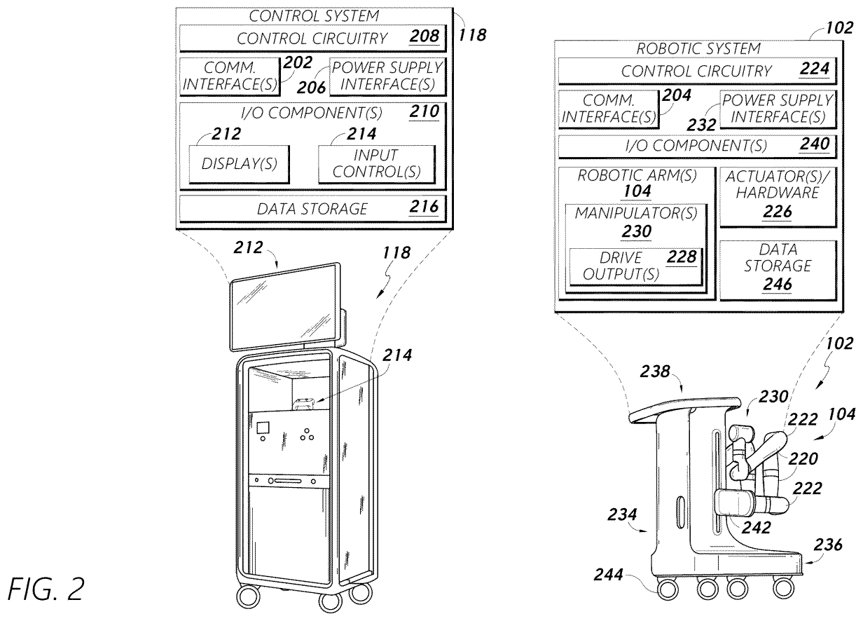

The medical system 100 can also include a control system 118 (also referred to as “control tower 118” or “mobile tower 118”), described in detail below with respect to FIG. 2. The control system 118 can be communicatively coupled (e.g., via wired and/or wireless connection(s)) to the robotic system 102 to provide support for controls, electronics, fluidics, optics, sensors, and/or power to the robotic system 102. Placing such functionality in the control system 118 can allow for a smaller form factor of the robotic system 102 that may be more easily adjusted and/or re-positioned by an operating user. Additionally, the division of functionality between the robotic system 102 and the control system 118 can reduce operating room clutter and/or facilitate efficient clinical workflow.

The medical system 100 can include an electromagnetic (EM) field generator 120, which is configured to broadcast/emit an EM field that is detected by EM sensors, such as a sensor associated with the medical instrument 106. The EM field can induce small currents in coils of EM sensors (also referred to as “position sensors”), which can be analyzed to determine a position and/or angle/orientation of the EM sensors relative to the EM field generator 120. The EM field generator 120 can be positioned to the side of the table 112 (as shown in FIG. 1), positioned under the table 112, positioned above the table 112, and so on. Although EM fields and EM sensors are described in many examples herein, position sensing systems and/or sensors can be any type of position sensing systems and/or sensors, such as optical position sensing systems/sensors, image-based position sensing systems/sensors, etc.

The medical system 100 can further include an imaging system(s) 122 (also referred to as “imaging device 122”) configured to generate and/or provide/send image data (also referred to as “image(s)”) to another device/system. For example, the imaging system(s) 122 can generate image data depicting anatomy of the patient 110 and provide the image data to the control system 118, robotic system 102, and/or another device. The imaging system(s) 122 can comprise an emitter/energy source (e.g., X-ray source) and/or detector (e.g., X-ray detector) mounted on a support 124 (which can include a C-shaped support or another shape), allowing for flexibility in positioning around the patient 110 to capture images from various angles without moving the patient 110. Use of the imaging system(s) 122 can provide visualization of internal structures/anatomy, which can be used for a variety of purposes, such as navigation of the medical instrument 106 (e.g., providing images of internal anatomy to a user), localization of the medical instrument 106 (e.g., based on an analysis of image data), etc. In examples, use of the imaging system(s) 122 can enhance the efficacy and/or safety of a medical procedure, such as a bronchoscopy, by providing clear, continuous visual feedback to the operating surgeon/team.

In some examples, the imaging system(s) 122 is a mobile device configured to move around within an environment. For instance, the imaging system(s) 122 can be positioned next to the patient 110 (as illustrated) during a particular phase of a procedure and removed when the imaging system(s) 122 is no longer needed. In other examples, the imaging system(s) 122 can be part of the table 112 or other equipment in an operating environment. The imaging system(s) 122 can be implemented as a Computed Tomography (CT) machine/system, X-ray machine/system, fluoroscopy machine/system, Positron Emission Tomography (PET) machine/system, PET-CT machine/system, CT angiography machine/system, Cone-Beam CT machine/system, 3DRA machine/system, single-photon emission computed tomography (SPECT) machine/system, Magnetic Resonance Imaging (MRI) machine/system, Optical Coherence Tomography (OCT) machine/system, ultrasound machine/system, etc. In some cases, the medical system 100 includes multiple imaging system, such as a first type of imaging system and a second type of imaging system, wherein the different types of imaging systems can be used or positioned over the patient 110 during different phases/portions of a procedure depending on the needs at that time.

In some examples, the imaging system(s) 122 is configured to process/generate multiple images (also referred to as “image data,” in some cases) to generate a three-dimensional (3D) view(s)/model. For example, the imaging device 122 can be implemented as a CT machine configured to capture/generate a series of images/image data (e.g., 2D images/slices) from different angles around the patient 110, and then use one or more algorithms to reconstruct these images/image data into a 3D model. The 3D model can be provided to the control system 118, robotic system 102, and/or another device, such as for processing, display, or otherwise. In examples, the imaging system(s) 122 is configured to generate 2D or 3D image data.

In examples, image data from the imaging system(s) 122 is used to localize various elements, such as the medical instrument 106, a target within anatomy, specific anatomical features, etc. For example, the control system 118 can be configured to provide navigation information (also referred to as “navigation data”) during a procedure to assist a user navigating the medical instrument 106 within the anatomy to reach a target (e.g., desired anatomical site/location). In some examples, a target can include a nodule, such as in the context of certain bronchoscopy procedures. To illustrate, the control system 118 can display a navigation view/graphical data 126 that includes an instrument indicator 128 representing the medical instrument 106, a target indicator 130 representing the target, and an anatomical map. The navigation data 126(A) (e.g., initial navigation data) can be determined based on sensor data from a sensor of the medical instrument 106 (e.g., EM sensor data associated with the EM field generator 120), a map of the anatomy, and/or a location of the target. In examples, the map and/or location of the target are based on preoperative data, such as data obtained during a preoperative phase to identify a target location and/or map the anatomy.

In any event, the navigation data 126(A) can be updated, if needed, based on image data 132 from the imaging system(s) 122. For example, the control system 118 can receive the image data 132 and analyze the image data 132 to determine a current/actual spatial relationship between the medical instrument 106 and the target. In examples, the control system 118 can display the image data 132 to a user, receive user input indicating a position of the medical instrument 106 and/or a position of the target in the image data 132, and analyze the image data 132 based on the user input to determine the current/intraoperative spatial relationship of the medical instrument 106 relative to the target. In some cases, an update operation is performed at 134 to update navigation if the control system 118 determines a difference between the location of instrument 106 (e.g., relative to the preoperative map or target) as indicated in the navigation data 126(A) and the location of the instrument 106 (e.g., relative to the target) as depicted in the image data 132. In performing the update operation at 134, the control system 118 can update the navigation data 126(A) and provide updated navigation data 126(B) that reflects the actual/real-time position of the medical instrument 106 relative to the target and/or the map. In examples, the location of instrument 106 as indicated in the navigation data 126(A) and the location of the instrument 106 as depicted in the image data 132 are compared in the same coordinate system/frame/space. Further, in some cases, an update operation is performed at 134 based on the location of the instrument 106 as depicted in the image data 132 without determining if there is a difference between the location of instrument 106 (e.g., relative to the preoperative map or target) as indicated in the navigation data 126(A) and the location of the instrument 106 (e.g., relative to the target) as depicted in the image data 132. Here, the navigation 126(A) may not actually change, such as in cases where there is no difference, even though the update operation is performed.

The various components of the medical system 100 can be communicatively coupled to each other over a network, which can include a wireless and/or wired network. Example networks include one or more personal area networks (PANs), local area networks (LANs), wide area networks (WANs), Internet area networks (IANs), cellular networks, the Internet, personal area networks (PANs), body area network (BANs), etc. In some examples, various communication interfaces can include wireless technology, such as Bluetooth, Wi-Fi, near-field communication (NFC), or the like. Furthermore, in some examples, the various components of the medical system 100 can be connected for data communication, fluid exchange, power exchange, and so on, via one or more support cables, tubes, connections, or the like.

FIG. 2 illustrates example components of the control system 118 and robotic system 102 in accordance with one or more examples. In various examples, the control system 118 and the robotic system 102 are implemented as a tower and a robotic cart, respectively. However, the control system 118 and robotic system 102 can be implemented in other manners. The control system 118 can be coupled to the robotic system 102 and operate in cooperation therewith to perform a medical procedure. For example, the control system 118 can include communication interface(s) 202 for communicating with communication interface(s) 204 of the robotic system 102 via a wireless or wired connection (e.g., to control the robotic system 102). Further, in examples, the control system 118 can communicate with the robotic system 102 to receive position/sensor data therefrom relating to the position of sensors associated with an instrument/member controlled by the robotic system 102. In some examples, the control system 118 can communicate with the EM field generator 120 to control generation of an EM field in an area around a patient. The control system 118 can further include a power supply interface(s) 206.

The control system 118 can include control circuitry 208 configured to cause one or more components of the medical system 100 to actuate and/or otherwise control any of the various system components, such as carriages, mounts, arms/positioners, medical instruments, imaging devices, position sensing devices, sensor, etc. Further, the control circuitry 208 can be configured to perform other functions, such as cause display of information, process data, receive input, communicate with other components/devices, and/or any other function/operation discussed herein.

The control system 118 can further include one or more input/out (I/O) components 210 configured to assist a physician or others in performing a medical procedure. For example, the one or more I/O components 210 can be configured to receive input and/or provide output to enable a user to control/navigate the medical instrument 106, the robotic system 102, and/or other instruments/devices associated with the medical system 100. The control system 118 can include one or more displays 212 to provide/display/present various information regarding a procedure. For example, the one or more displays 212 can be used to present navigation information including a virtual anatomical model of anatomy with a virtual representation of a medical instrument, image data, and/or other information. The one or more I/O components 210 can include a user input control(s) 214, which can include any type of user input (and/or output) devices or device interfaces, such as one or more buttons, keys, joysticks, handheld controllers (e.g., video-game-type controllers), computer mice, trackpads, trackballs, control pads, sensors (e.g., motion sensors or cameras) that capture hand gestures and finger gestures, touchscreens, toggle (e.g., button) inputs, and/or interfaces/connectors therefore. In examples, such input(s) can be used to generate commands for controlling medical instrument(s), robotic arm(s), and/or other components.

The control system 118 can also include data storage 216 configured to store executable instruments (e.g., computer-executable instructions) that are executable by the control circuitry 208 to cause the control circuitry 208 to perform various operations/functionality discussed herein. In examples, two or more of the components of the control system 118 can be electrically and/or communicatively coupled to each other.

The robotic system 102 can include the one or more robotic arms 104 configured to engage with and/or control, for example, the medical instrument 106 and/or other elements/components to perform one or more aspects of a procedure. As shown, each robotic arm 104 can include multiple segments 220 coupled to joints 222, which can provide multiple degrees of movement/freedom. The robotic system 102 can be configured to receive control signals from the control system 118 to perform certain operations, such as to position one or more of the robotic arms 104 in a particular manner, manipulate an instrument, and so on. In response, the robotic system 102 can control, using control circuitry 224 thereof, actuators 226 and/or other components of the robotic system 102 to perform the operations. For example, the control circuitry 224 can control insertion/retraction, articulation, roll, etc. of a shaft of the medical instrument 106 or another instrument by actuating a drive output(s) 228 of a manipulator(s) 230 (e.g., end-effectors) coupled to a base of a robotically-controllable instrument. The drive output(s) 228 can be coupled to a drive input on an associated instrument, such as an instrument base of an instrument that is coupled to the associated robotic arm 104. The robotic system 102 can include one or more power supply interfaces 232.

The robotic system 102 can include a support column 234, a base 236, and/or a console 238. The console 238 can provide one or more I/O components 240, such as a user interface for receiving user input and/or a display screen (or a dual-purpose device, such as a touchscreen) to provide the physician/user with preoperative and/or intraoperative data. The support column 234 can include an arm support 242 (also referred to as “carriage 234”) for supporting the deployment of the one or more robotic arms 104. The arm support 242 can be configured to vertically translate along the support column 234. Vertical translation of the arm support 242 allows the robotic system 102 to adjust the reach of the robotic arms 104 to meet a variety of table heights, patient sizes, and/or physician preferences. The base 236 can include wheel-shaped casters 244 (also referred to as “wheels 244”) that allow for the robotic system 102 to move around the operating room prior to a procedure. After reaching the appropriate position, the casters 244 can be immobilized using wheel locks to hold the robotic system 102 in place during the procedure.

The joints 222 of each robotic arm 104 can each be independently-controllable and/or provide an independent degree of freedom available for instrument navigation. In some examples, each robotic arm 104 has seven joints, and thus provides seven degrees of freedom, including “redundant” degrees of freedom. Redundant degrees of freedom can allow robotic arms 104 to be controlled to position their respective manipulators 230 at a specific position, orientation, and/or trajectory in space using different linkage positions and joint angles. This allows for the robotic system 102 to position and/or direct a medical instrument from a desired point in space while allowing the physician to move the joints 222 into a clinically advantageous position away from the patient to create greater access, while avoiding collisions.

The one or more manipulators 230 (e.g., end-effectors) can be couplable to an instrument base/handle, which can be attached using a sterile adapter component in some instances. The combination of the manipulator 230 and coupled instrument base, as well as any intervening mechanics or couplings (e.g., sterile adapter), can be referred to as a manipulator assembly, or simply a manipulator. Manipulator/manipulator assemblies can provide power and/or control interfaces. For example, interfaces can include connectors to transfer pneumatic pressure, electrical power, electrical signals, and/or optical signals from the robotic arm 104 to a coupled instrument base. Manipulator/manipulator assemblies can be configured to manipulate medical instruments (e.g., surgical tools/instruments) using techniques including, for example, direct drives, harmonic drives, geared drives, belts and/or pulleys, magnetic drives, and the like.

The robotic system 102 can also include data storage 246 configured to store executable instruments (e.g., computer-executable instructions) that are executable by the control circuitry 224 to cause the control circuitry 224 to perform various operations/functionality discussed herein. In example, two or more of the components of the robotic system 102 can be electrically and/or communicatively coupled to each other.

Data storage (including the data storage 216, data storage 246, and/or other data storage/memory) can include any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data.

Computer-readable media that can include, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

Control circuitry (including the control circuitry 208, control circuitry 224, and/or other control circuitry) can include circuitry embodied in a robotic system, control system/tower, instrument, or any other component/device. Control circuitry can include any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein can further include one or more circuit substrates (e.g., printed circuit boards), conductive traces and vias, and/or mounting pads, connectors, and/or components. Control circuitry can further comprise one or more storage devices, which may be embodied in a single device, a plurality of devices, and/or embedded circuitry of a device. Such data storage can comprise read-only memory, random access memory, volatile memory, non-volatile memory, static memory, dynamic memory, flash memory, cache memory, data storage registers, and/or any device that stores digital information. In examples in which control circuitry comprises a hardware and/or software state machine, analog circuitry, digital circuitry, and/or logic circuitry, data storage device(s)/register(s) storing any associated operational instructions can be embedded within, or external to, the circuitry comprising the state machine, analog circuitry, digital circuitry, and/or logic circuitry.

Functionality described herein can be implemented by the control circuitry 208 of the control system 118 and/or the control circuitry 224 of the robotic system 102, such as by the control circuitry 208, 224 executing executable instructions to cause the control circuitry 208, 224 to perform the functionality.

### Mapping, Navigation, and Positioning Modalities/Systems

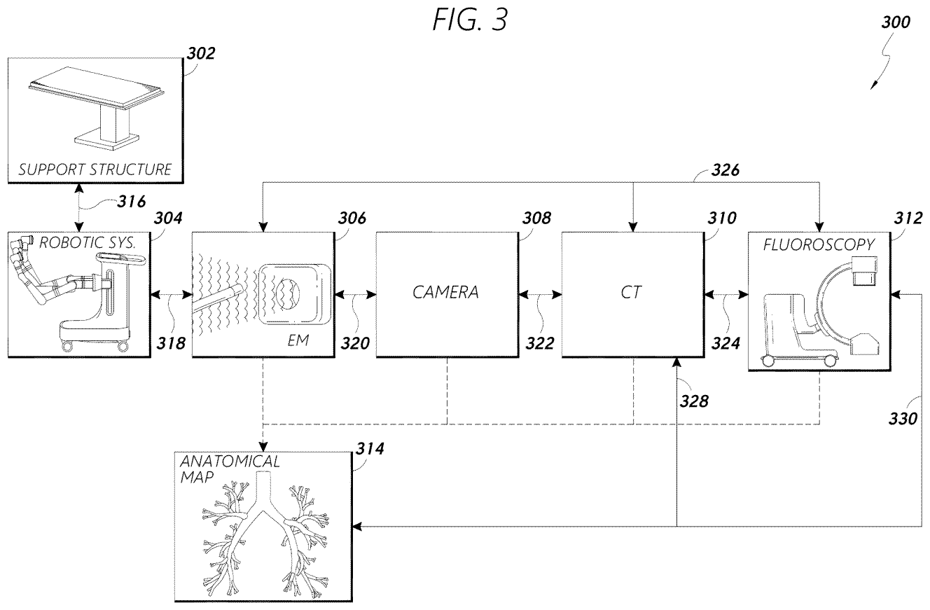

FIG. 3 is a block diagram illustrating a system 300 including various positioning and/or imaging systems/modalities 302-312 (sometimes referred to as “subsystems 302-312”), which can be implemented to facilitate anatomical mapping, navigation, positioning, and/or visualization for procedures in accordance with one or more examples. For example, the various systems 302-312 can be configured to provide data for generating an anatomical map, determining a location of an instrument, determining a location of a target, and/or performing other techniques.

Each of the systems 302-312 can be associated with a respective coordinate frame (also referred to as “position coordinate frame’) and/or can provide data/information relating to instrument and/or anatomy locations, wherein registering the various coordinate frames to one another can allow for integration of the various systems to provide mapping, navigation, and/or instrument visualization. For example, registration of various modalities to one another can allow for determined positions in one modality to be tracked and/or superimposed on/in a reference frame associated with another modality, thereby providing layers of positional information that can be combined to provide a robust localization system.

In examples, the system 300 is configured to implement one or more localization/localizing techniques (also referred to as “localization/localizing system 300”). Localization/localizing can refer to processes of determining a location and orientation/pose of an instrument or other element/component within a given space or environment.

In various examples, the anatomical space in which a medical instrument can be localized (i.e., where position and/or shape of the instrument is determined/estimated) is a 2D or 3D portion of a patient's tracheobronchial airways, vasculature, urinary tract, gastrointestinal tract, or any organ or space accessed via lumens. Various modalities can be implemented to provide images/representations/models of the anatomical space. For example, an imaging modality can be implemented, which can include, for example, X-ray, fluoroscopy, CT, Positron Emission Tomography (PET), PET-CT, CT angiography, Cone-Beam CT (CBCT), 3DRA, single-photon emission computed tomography (SPECT), Magnetic Resonance Imaging (MRI), Optical Coherence Tomography (OCT), and ultrasound. One or both of preprocedural and intraprocedural images can be acquired in connection with a procedure.

The systems 302-312 can provide information for generating a 2D or 3D anatomical model/map 314 (e.g., airway model). In examples, the anatomical map 314 and/or other localization information can be displayed to a user, such as a physician, during a procedure to assist the user in performing the procedure. For example, a visualization of a tracked instrument can be superimposed on the anatomical map 314 based on position/sensor data associated with the tracked medical instrument.

As shown, the system 300 can include a surgical bed or other patient platform or positioning/support structure 302 (referred to as “support structure 302” for convenience). The position of the support structure 302 can be known based on data maintained relating to the position of the support structure 302 within the surgical/procedure environment. Alternatively, or additionally, the position of the support structure 302 can be sensed or otherwise determined using one or more markers and/or an appropriate imaging/positioning modality.

The system 300 can further include a robotic system 304, such as the robotic system 102 (e.g., a robotic cart or other device or system including one or more robotic end effectors). Data relating to the position and/or state of robotic arms, actuators, and/or other components of the robotic system 304 can be known or derived from robotic command data or other robotic data relative to a coordinate frame of the robotic system 304. In some examples, reference frame registration 316 occurs between the support structure 302 and the robotic system 304, which can be a relatively coarse registration (in some cases) based on robotic system/cart-set-up procedure (which can have any suitable or desirable scheme).

The system 300 can further include an electromagnetic (EM) sensor system 306, which can include an EM field generator (e.g., the EM field generator 120) and one or more EM sensors. An EM sensor can be associated with a portion of an instrument that is tracked/controlled, such as along a length of the instrument and/or another elongate member disposed in the working channel of the instrument. In some implementations, the EM field generator can be mechanically coupled to either the support structure 302 or the robotic system 304, in which case registration/association 318 between such systems can be known and/or determined. In some implementations, the registration 318 between the EM sensor system 306 and the robotic system 304 can be determined through forward kinematics and/or field generator mount transform information. For example, the field generator can be mounted to an end effector/manipulator of the robotic system 304, such that the position of the field generator can be known relative to the robotic system positioning frame based on the known relationship between the position of the robotic end effector and the robotic system 304. The EM sensor system 306 can provide instrument pose and/or path information based on sensor readings associated with the instrument.

The system 300 can further include an optical camera system 308 including one or more cameras or other imaging devices, wherein such device(s) is/are configured to generate images of patient anatomy within a visual field thereof, such as real-time image data during a surgical procedure. In examples, registration 320 between the optical camera system 308 and the EM sensor system 306 can be achieved through identification of features having EM sensor data associated therewith, such as by a medical instrument tip, in images generated by the optical camera system 308. The registration 320 can further be based at least in part on hand-eye interaction of the physician when viewing real-time camera images while the EM-sensor-equipped endoscope is navigating in the patient anatomy.

The system 300 can further include a computed tomography (CT) imaging system 310 configured to generate CT images of the patient anatomy, which can be done preoperatively and/or intraoperatively. In examples, image processing can be implemented for registration 322 of the CT image data with the camera image data generated by the optical camera system 308. For example, common features identified in both camera image data and CT image data can be identified to relate the CT image frame to the camera image frame in space. In some examples, the CT imaging system 310 can be used to generate preoperative imaging data for producing the anatomical map 314 and/or for path navigation planning.

The system 300 can further include a fluoroscopy imaging system 312 configured to generate X-ray images (e.g., 2D real-time images) of the surgical site. In some examples, preprocedural and/or intraprocedural images are acquired using a C-arm fluoroscope. The fluoroscopy imaging system 312 can be used with a contrast agent introduced into the anatomy to generate image data representing patient anatomy and/or instrumentation. In examples, the fluoroscopy imaging system 312 can be registered 324 to the CT imaging system 310 using any image processing technique suitable for such registration.

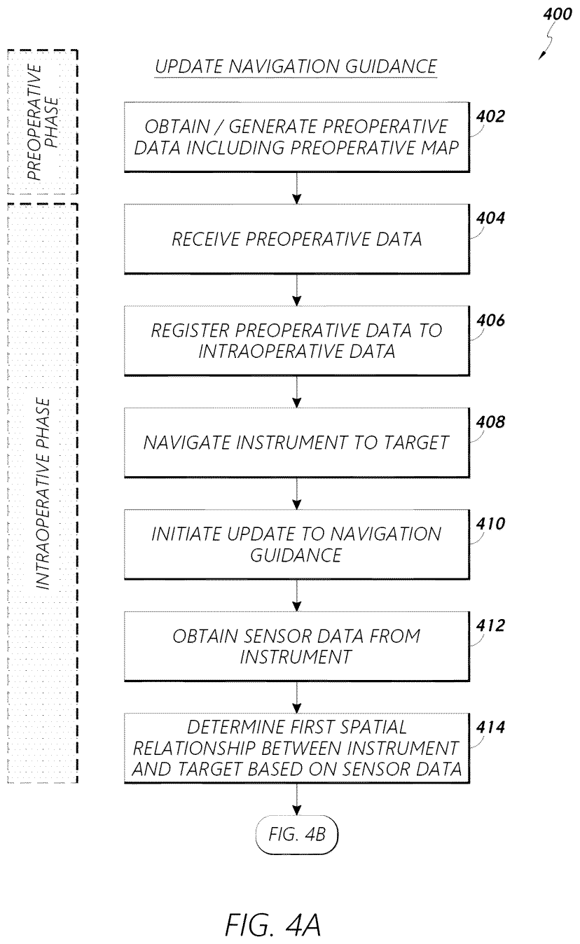

In examples, the CT imaging system 310 and/or the fluoroscopy imaging system 312 can be registered 326 to the EM sensor system 306 through various techniques, such as tool registration, a transformation function, etc. In one example, a mechanical structure of the C-arm instrumentation of the system 310, 312 can have a known physical transform/relationship with respect to a mounting position of the EM field generator of the EM sensor system 306. Such known relationship can be used to register the CT/fluoroscopy image space to the EM sensor image space. Further, in another example, the registration 326 includes performing other operations discussed herein, such as those discussed in reference to FIG. 4. The connections 328, 330 represent registrations/relationships of the CT imaging system 310 and the fluoroscopy imaging system 312 to the anatomical map 314, respectively.

Although the CT imaging system 310 and fluoroscopy imaging system 312 are illustrated as separated systems, in examples the same system performs the functionality of the CT imaging system 310 and fluoroscopy imaging system 312.

The position, shape, and/or orientation of an instrument, such as an endoscope, can be determined using any one or more of the systems 302-312, which can facilitate generation of graphical interface data representing the estimated position and/or shape of the instrument relative to the anatomical map 314. The position, shape, and/or orientation of the instrument and/or anatomical map 314 can be displayed on a display device, such as via the control system 118 and/or robotic system 102, or another device. In some examples, the anatomical map 314 also indicates a position(s) of a target(s), such as a location within the anatomy that has been designated for further treatment.

Although the systems 302-312 are discussed in a specific order, the systems can be implemented in different orders. Moreover, the systems can be used in different ways. Further, registration can occur between different systems.

In examples, any number of coordinate frames/spaces of the systems 302-312 can be registered/aligned to represent/associate data from multiple coordinate frames in the same coordinate frame. In some cases, the coordinate frame that is used may be a specific coordinate frame, while in other cases the coordinate frame that is used may be less relevant. In examples, preoperative data and intraoperative data can be represented within the same coordinate frame/space. To illustrate, preoperative data associated with the CT imaging system 310, preoperative data associated with the fluoroscopy system 312, intraoperative data associated with the EM sensor system 306, intraoperative data associated with the CT imaging system 310, and/or intraoperative data associated with the fluoroscopy system 312 can be associated/represented within the same coordinate frame, such as in intraoperative coordinate frame.

In some illustrations, one or more of the systems 302-312 can be implemented to generate the anatomical map 314 preoperatively (or intraoperatively) and/or determine a location of one or more targets within the anatomical map 314. Intraoperatively, one or more of the systems 302-312 can also be implemented to determine a location of a medical instrument and/or position of a target relative to the anatomical map 314. As discussed herein, one or more of the systems 302-312 can also be implemented to update the anatomical map 314, location of the medical instrument, location of the target, etc.

### Example Flow Diagrams

FIGS. 4, 5, and 6 illustrate example flow diagrams of processes 400, 500, and 600, respectively, for facilitating various operations/acts in accordance with one or more examples discussed herein. One or more of the acts/operations associated with the processes 400, 500, and/or 600 can be performed by control circuitry implemented in any of the devices/systems discussed herein or a combination thereof, such as the control circuitry 208 of the control system 118, the control circuitry 224 of the robotic system 102, etc. Although various blocks/operations are illustrated in a particular order, the blocks/operations can be implemented in other orders (e.g., arranged differently). Further, any of the blocks/operations can be eliminated.

FIGS. 4A and 4B illustrate an example flow diagram of the process 400 for updating navigation guidance in accordance with one or more examples.

In FIG. 4A, at block 402, the process 400 includes obtaining or generating preoperative data (also referred to as “preprocedural data”), which can include a preoperative map of anatomy (also referred to as the “anatomical map”). For example, control circuitry can be configured to generate a preoperative map of anatomy based on any of the techniques discussed herein or other techniques, such as any of the techniques discussed in reference to FIG. 3. To illustrate, during a preoperative phase (e.g., prior to an operative procedure), image data can be captured of a patient's anatomy using one or more imaging techniques (e.g., CT imaging, MRI imaging, etc.). The image data can be processed to generate an anatomical map, which can include/indicate various pathways/lumens and/or other features within the anatomy. Further, the preoperative data can include information about a target(s) within the anatomy, such as a location designated within the anatomy as a target, a size of the target, etc. In examples, the target can be designated based on user input tagging/specifying a location, an analysis of the image data to identify an object/target that includes certain characteristics, etc. Moreover, the preoperative data can indicate a location(s) of a certain/predetermined anatomical feature(s). As such, the preoperative data can indicate a preoperative location of the target/anatomical feature within the anatomy, such as the location of the target/anatomical feature relative to the map, the location of the target/anatomical feature relative to a coordinate frame (sometimes referred to as a “preoperative coordinate frame”), etc. Although block 402 is discussed in the context of being performed during a preoperative phase, block 402 can be performed during an intraoperative phase and/or at other times to generate an anatomical map, identify a location of a target, etc.

At bock 404, the process 400 includes receiving preoperative data regarding anatomy associated with a procedure. For example, during an intraoperative phase, control circuitry can be configured to receive preoperative data generated during a preoperative phase, such as the preoperative data generated in block 402. As noted above, the preoperative data can include information regarding an anatomical map, target within the anatomy, certain anatomical features, etc. In examples, preoperative data is generated several days, weeks, etc. before an intraoperative phase. However, preoperative data can be generated at other times.

At block 406, the process 400 includes registering/aligning the preoperative data to intraoperative data. For example, control circuitry can be configured to register/align a preoperative map, coordinate frame/space associated with the preoperative data, anatomical features designated with the preoperative map, and/or location(s) of target(s) within the preoperative map to an intraoperative map, a coordinate frame/space associated with the intraoperative environment (e.g., a coordinate frame used to navigate an instrument intraoperatively), anatomical features identified intraoperatively, etc. To illustrate, the intraoperative map can be associated with an EM space/coordinate frame used to track an instrument intraoperatively.

In some examples, the registration process includes navigating an instrument intraoperatively to certain/known anatomical features within the anatomy that are identified within preoperative data. Locations of the instrument can be determined/tracked as the instrument navigates to the anatomical features. Here, preoperative locations for the anatomical features can be mapped to the corresponding intraoperative locations of those anatomical features, thereby registering the preoperative map to the current/intraoperative coordinate frame/space. Further, in some examples, other techniques are used to register the preoperative data to the intraoperative data, which can include using various types of intraoperative data, such as position data of an instrument, image data from an instrument, image data from an external imaging system, features/locations identified based on image analysis, fiducial markers placed on a patient in proximity to certain anatomical features, fiducial markers on a table/support or another object, etc.

In examples, by registering/aligning preoperative data to intraoperative data, the preoperative data and intraoperative data can be associated with the same coordinate frame, thereby allowing the current location of the instrument to be represented relative to preoperative data. To illustrate, a preoperative map and/or target can be registered/aligned to an intraoperative coordinate frame (or vice versa), wherein navigation information for an instrument is provided relative to the preoperative map. This can allow a user to view a current location of an instrument and/or preoperative location of the target relative to the preoperative map. However, as mentioned above, differences in the characteristics of the anatomy between preoperative and intraoperative phases, distortion introduced into an operating environment, and/or other factors can cause instrument error (e.g., inaccuracies in providing the current/intraoperative location of the instrument, since the instrument location is displayed relative to the preoperative map). In examples, the process 400 is performed to update/correct inaccuracies in the navigation information. However, the process 400 can be performed in other contexts.

At block 408, the process 400 includes navigating an instrument to a target. For example, control circuitry can be configured to control a robotic system based on user input to navigate an instrument coupled to the robotic system to a target. The user can provide input to navigate the instrument based on viewing navigation information (e.g., graphical interface data) that presents a real-time location of the instrument relative to an anatomical map and/or target, such as relative to a preoperative map. In examples, the instrument can be navigated to within a predetermined distance/proximity to the target and/or parked (e.g., movement stops). In any event, the instrument can be guided to a location believed to correspond to the target.

At block 410, the process 400 includes initiating an update to navigation guidance. In one example, control circuitry can be configured to receive user input requesting to initiate an update to navigation guidance (e.g., entering a menu and selecting a navigation update button). In response, the process 400 can continue to block 412. In another example, control circuitry can automatically determine to initiate an update to navigation guidance, such as when the instrument is positioned within a predetermined proximity to a target and remains stationary for more than a threshold amount of time or when another event occurs.

In some examples, navigation/movement of the instrument is paused/stopped/restricted upon initiating an update to navigation guidance. For example, after initiating an update to navigation guidance at block 410 or at other times, a notification/message can be displayed to the user (i) indicating that navigation/movement of the instrument is paused/stopped (e.g., the instrument is immobilized) or (ii) requesting that the user ceases to provide input to control the instrument. However, navigation of the instrument can be paused/stopped without providing a notification/message. In any event, pausing/stopping movement of the instrument can allow sensor data from the instrument to be collected (at block 412) or other operations to be performed while the instrument remains at a relatively fixed position. Further, pausing/stopping movement of the instrument can allow image data to be captured by the imaging system (at block 418 in FIG. 4B) with preserved/higher image quality, since image quality can be reduced by instrument motion. In some cases, movement/navigation of the instrument is stopped while one or more of blocks 412-430 are performed. However, one or more of the blocks of the process 400 can be performed without pausing/stopping/restricting movement of the instrument.

At block 412, the process 400 includes obtaining sensor data from the instrument. For example, control circuitry can be configured to receive sensor data from the instrument, wherein the sensor data indicates or is used to determine a position/orientation of the instrument, such as a position/orientation of a distal end of the instrument or another portion of the instrument where a sensor is located.

At block 414, the process 400 includes determining a first spatial relationship between an instrument and a target based on sensor data. For example, control circuitry can be configured to determine a position/orientation of the instrument relative to a location of the target based on the sensor data obtained at block 412. Here, the position/orientation of the instrument and the location of the target can be represented relative to a preoperative map (with the instrument position and preoperative map associated with the same coordinate frame). Thus, the first spatial relationship between the instrument and the target can be determined relative to the preoperative map.

In FIG. 4B, at block 416, the process 400 includes placing/positioning an imaging system in proximity to anatomy and/or connecting the imaging system. For example, a user can maneuver/move the imaging system to position at least a portion of the imaging system over a patient, such as with a C-arm of the imaging system over a region of the anatomy being examined/treated. In some cases, a notification/message can be displayed to request that the user position the imaging system in a particular manner, such as at a particular angle relative to a patient, patient table, etc. The user can connect/communicatively couple the imaging system to a control system, robotic system, etc. The imaging system can be located external to an internal anatomical site and configured to capture/generate image data depicting internal anatomy. For instance, the imaging system can be configured to capture/generate X-ray, CT, or other image data representing the internal anatomy.

Although block 416 is illustrated as part of the process 400, in some cases the imaging system is already positioned/placed at the appropriate position and/or connected to the appropriate devices/system, such that block 416 is not performed. In one example, the imaging system is part of a table/support structure on which a patient is positioned and/or already connected to a control/robotic system.

At block 418, the process 400 includes generating image data and/or receiving the image data from the imaging system. For example, the imaging system can generate/capture image data depicting internal anatomy (e.g., scan the patient) and/or send the image data to another component/device. For instance, control circuitry of a control/robotic system or another system can receive the image data from the imaging system. In examples, image data includes X-ray image data, fluoroscopy image data, CT image data, Positron Emission Tomography (PET) image data, PET-CT image data, CT angiography image data, Cone-Beam CT image data, 3DRA image data, single-photon emission computed tomography (SPECT) image data, Magnetic Resonance Imaging (MRI) image data, Optical Coherence Tomography (OCT) image data, ultrasound image data, etc.

In some examples, the imaging system is rotated/moved from an initial position/arrangement/orientation to an end position/arrangement/orientation while generating image data at block 418. For instance, upon completing the operation at block 418, a portion of the imaging system can be placed in a lateral position. This position can be less desirable for continuing with the process 400 and/or for performing other operations. For instance, this position can cause interference/distortion in obtaining sensor data from an instrument, make it more difficult to perform other portions of a procedure, make it less desirable for using the imaging system later, etc. In some cases, a message/notification can be provided to request that the user return the imaging system to a particular position/arrangement/orientation, such as an initial or upright position or other position out of the way. In some cases, user input is received indicating that the imaging system has been repositioned and the process 400 can proceed. Moreover, in some cases, a user can move the imaging system to another location away from the patient, such as to a different location with the environment.

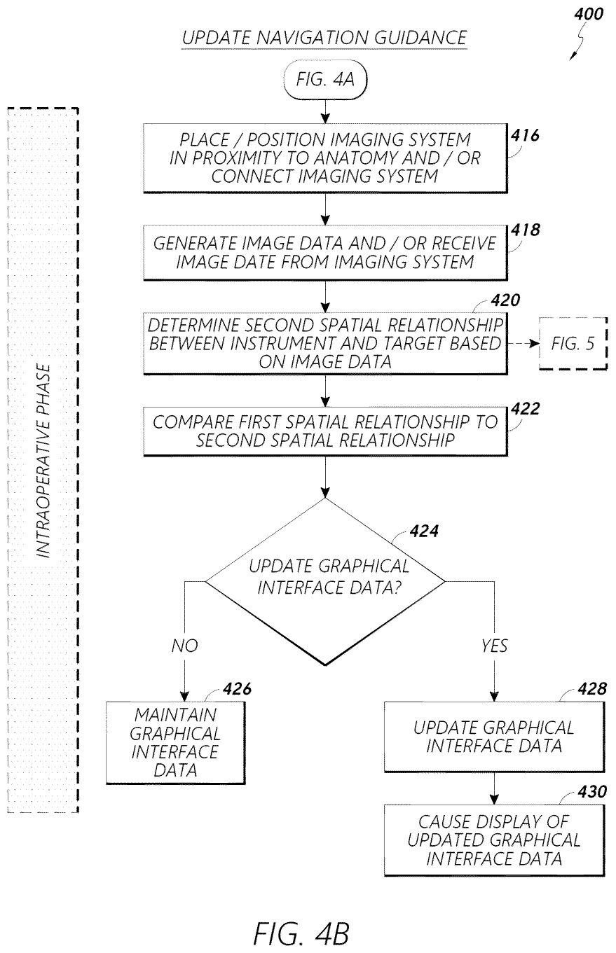

At block 420, the process 400 includes determining a second spatial relationship between the instrument and the target location based on the image data. In examples, control circuitry can be configured to determine the second spatial relationship based on user input that selects/identifies the instrument and/or the target in the image data. Further, in examples, control circuitry can determine the second spatial relationship by performing one or more image processing techniques (e.g., Artificial Intelligence (AI) techniques, machine-trained models, traditional image analysis/processing, etc.). In examples, one or more image processing techniques are performed automatically, such as without using user input. In other examples, user input is used with or without image processing. In any event, the second spatial relationship can include/represent a distance and/or vector between a distal end of the instrument and the target in the image data. The second spatial relationship can represent an intraoperative or current relationship between the instrument and the target. Example techniques for determining the second spatial relationship are discussed below in the context of the process 500 of FIG. 5. However, other processes/operations can be performed.

At block 422, the process 400 includes comparing the first spatial relationship to the second spatial relationship. In one example, the first spatial relationship and the second spatial relationship are each represented with a vector/line within a coordinate frame/space, wherein each vector/line represents/indicates a distance and direction from the target to the distal end of the instrument. For instance, a first vector (of the first spatial relationship) can represent a distance and direction of the instrument to the target within a coordinate frame/space. Whereas a second vector (of the second spatial relationship) can represent a distance and direction of the instrument to the target within the same coordinate frame/space. In cases where the first spatial relationship is represented with a first vector and the second spatial relationship is represented with a second vector, the first vector can be compared to the second vector to determine a difference in one or more parameters/values/characteristics between the first and second vectors. In doing so, a variety of vector comparison techniques/characteristics can be used/compared, such as Euclidian distance, dot product, cross product, Manhattan distance, angle between vectors, vector norm, projection, magnitude, direction, angle, etc. As such, in examples of block 422, a difference between first and second spatial relationships can be determined.

At block 424, the process 400 includes determining whether to update graphical interface data. For instance, in some cases, at block 424, control circuitry can determine if a difference between the first spatial relationship and the second spatial relationship satisfies one or more criteria, such as the difference being non-zero (e.g., a difference between one or more parameters/values/characteristics of the first spatial relationship and one or more parameters/values/characteristics of the second spatial relationship is non-zero by more than a threshold). However, one or more other criteria can be used.

If it is determined to not update the graphical interface data, the process 400 can proceed to block 426. Alternatively, if it is determined to update the graphical interface data, the process 400 can proceed to block 428.

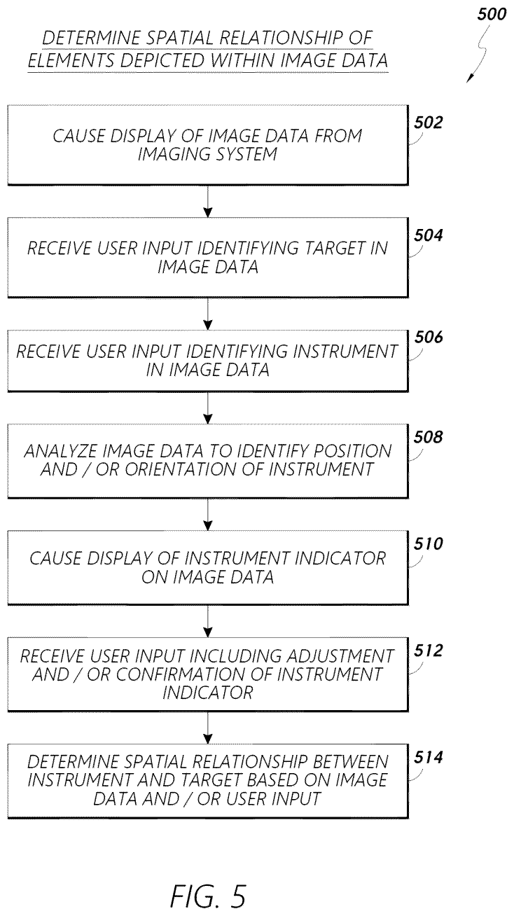

At block 426, the process 400 includes maintaining graphical interface data. For example, graphical interface data can be displayed representing an anatomical map, location of the instrument (based on the sensor data obtained from the instrument at block 412), and/or location of the target. Example techniques for providing graphical interface data are discussed below in reference to FIG. 6. At block 426, control circuitry can maintain the graphical interface data in an unchanged format, such as by continuing to provide/present the graphical interface data without changing the location of the instrument and/or location of the target.

At block 428, the process 400 includes updating graphical interface data. For example, control circuitry can be configured to update graphical interface data representing an anatomical map, location of the instrument, and/or location of the target such that the updated graphical interface data indicates the second spatial relationship between the instrument and the target. In some cases, the update is based on the second spatial relationship and/or a difference between the first and second spatial relationships. The update can include moving the location of the instrument (e.g., moving an instrument indicator), moving a location of the target (e.g., moving a target indicator), shifting/updating/editing/replacing the anatomical map, and/or otherwise updating the graphical interface data. In examples, a message/notification can be displayed to a user to confirm that the user desires to update the graphical interface data. In response to receiving user input including a confirmation to update the graphical interface data, the graphical interface data can be updated at block 428. As such, by updating the graphical interface data, navigation information for the instrument can be updated.

At block 430, the process 400 includes causing display of the updated graphical interface data. For example, control circuitry can cause the updated graphical interface data to be displayed via a display associated with a control system, robotic system, or other system. In examples, causing display of the updated graphical interface data includes displaying the graphical interface data via a display/display screen. In other examples, causing display of the updated graphical interface data includes sending/providing the updated graphical interface data to a display/display device for the display/display device to display the updated graphical interface data (e.g., content). In some instances, control circuitry can enable a user to provide input to undo an update to graphical interface data (e.g., revert back to previous/initial graphical interface data) and/or to redo an update to graphical interface data (e.g., provide new user input to designate a location of an instrument/target within image data and determine the second spatial relationship again).

Although the process 400 illustrates blocks 424 and 426, in examples the process 400 can proceed to update graphical interface data (at block 428) without performing the operation of block 424 (and/or the operation of block 426). In one illustration, the process 400 can proceed from block 422 without implementing blocks 424 and/or 426. For instance, the process 400 can include updating graphical interface data (at block 428) upon determining a difference between the first and second spatial relationships (at block 422). Here, at block 428, the process 400 can include updating the graphical interface data based on the second spatial relationship and/or a difference between the first and second spatial relationships. In some cases, the operation at block 428 can be performed even if there is no actual change to the graphical interface data upon completing block 428 (e.g., the difference between the first and second spatial relationships is zero). As such, in some cases, block 424 may not be performed.

Although various examples are discussed in the context of graphical interface data that is displayable, the techniques discussed herein can alternatively or additionally generate/update navigation data that is non-displayable and/or used for other purposes (e.g., navigation data used to track a location of an instrument, which may or may not be displayable).

In examples, error management operations/techniques (also referred to as “error handling techniques”) are implemented to detect and/or address errors/faults/issues that occur during the process 400 (or other processes discussed herein). In some cases, the error management techniques include providing one or more messages/notifications (also referred to as “an error message” or “warning message”) including information about an error/warning (e.g., a type of error/warning or other details about the error/warning, a corrective action to address the error/warning, etc.), a request that the user perform a particular action/act to address the error/warning, etc. An error/warning message can be displayed to the user, provided via audio output, provided via haptic output, and/or other types of output. A user can view an error/warning message and provide user input to confirm that the error/warning message has been viewed, confirm that the user has performed a corrective action, initiate a corrective action, etc. In some cases, handling/addressing an error/warning includes returning to a block in the process 400 to start again from a particular point in the process 400 or to perform an operation again that is associated with a particular block before moving on in the process 400. The error management techniques can generally be performed by control circuitry.

Although example error management techniques can generally provide an error/warning message to a user, in some cases the error management techniques automatically handle an error/warning without providing an error/warning message and/or without receiving user input from a user regarding the error/warning. Further, although various errors are discussed herein as being triggered at certain times during the process 400, the errors/warning can be triggered at other times. Moreover, although examples are discussed in the context of providing error/warning messages and proceeding when the errors/warnings are addressed (e.g., a user providing input indicating that an error/warning has been resolved, a detection that the error/warning has been resolved, etc.), in some cases the process 400 proceeds as normal upon providing an error/warning message.

In examples, an instrument attachment error (also referred to as “an instrument unloaded error”) is triggered when the instrument is decoupled/detached from a robotic system. For instance, control circuitry can be configured to detect when the instrument (and/or other components associated with the instrument, such as an access sheath) is removed/detached from a robotic arm/component of a robotic system while any block of process 400 is performed. In response, the control circuitry can provide a notification to reconnect/reattach the instrument to the robotic system and a message that certain operations will be performed again once the instrument is reconnected. To illustrate, if the instrument is removed from the robotic system while one or more of blocks 410-430 are performed, the process 400 can exit a navigation guidance update (e.g., initiated at block 410), return to block 410 and restart the process 400 from that point, etc. In some cases, the process 400 may not restart until it is detected that the instrument is reconnected to the robotic system.

In examples, an instrument sensor error is triggered when there are issues with sensor data or a sensor of an instrument. For instance, an EM sensor or another sensor on an instrument can experience distortion, loss/reduction of sensing an EM field, a malfunction, or other issues. This can degrade the quality of the sensor data. In some cases, moving the imaging system in proximity to a patient before completing block 412 can cause distortion or other issues. In one example, control circuitry can be configured to detect (at/after block 412) that sensor data is associated with more than a threshold amount of distortion, more than a threshold amount of reduction in quality, etc. Additionally or alternatively, control circuitry can be configured to detect (at/after block 412) a loss/reduction of receiving sensor data (e.g., less than a particular number of EM samples/readings are received over a predetermined period of time). In any event, in response to such detection, the control circuitry can provide a notification to remove a source of the distortion and/or reinitiate an update to navigation guidance. Here, the process 400 can restart at block 412 to recapture sensor data once the distortion source is removed and/or the user input is provided to restart. In another example, control circuitry can be configured to (at/after block 420) make a similar detection, provide a notification regarding the instrument sensor error, and restart at block 412. In other examples, an instrument sensor error is triggered at other times during the process 400. Although various examples are discussed in the context of restarting at block 412 to recapture sensor data, in some cases a warning notification is provided indicating that relatively low-quality image data is obtained and will be used. Here, the process 400 can continue as normal without returning to a previous block. Further, in some cases, instead of restarting at block 412 to recapture sensor data, the process 400 can exit a navigation guidance update (e.g., initiated at block 410), requiring a user/system to reinitiate an update to navigation guidance when desired.

In examples, an instrument sensor warning/error is triggered when there are issues with sensor data. For instance, if an imaging system is left at an end position (e.g., lateral position) upon completing the operation at block 418, the imaging system can interfere with obtaining sensor data from an instrument, as noted above. In some cases, sensor data is received from the instrument during an update to graphical interface data at block 428. As such, a warning/error message can be provided indicating that the quality of sensor data is poor/low and/or requesting to position/move the imaging system to a particular position/arrangement/orientation (e.g., an initial or another position/arrangement/orientation that is out of the way) when it is detected that sensor data is associated with more than a threshold amount of distortion, sensor data does not satisfy one or more quality criteria, sensor data is not received over a period of time (e.g., less than a particular number of EM samples/readings are received over a predetermined period of time), etc. Although examples are discussed in the context of the imaging system potentially causing interference, other objects/items can cause undesirable effects, wherein the warning/error message can request that any object that may potentially effect sensor data be moved from the space.

In examples, an imaging system error (sometime referred to as an “image data error”) is triggered when there are issues with image data or an imaging system configured to capture the image data. In one example, control circuitry can be configured to detect (at/before/after block 416/418 or any other time) that an imaging system is not connected or properly connected to a control/robotic system, such as by detecting that a signal is not received from the imaging system. In response, the control circuitry can provide a notification indicating that the imaging system is not detected/connected. The notification can provide a request to check a network connection, reconnect the imaging system, and/or recapture image data with the imaging system (perform block 416/418 again). In response to receiving user input confirming that the imaging system is reconnected, the control circuitry can return to block 416/418. Although examples of image system errors are discussed in the context of restarting at block 416/418, in some cases a warning notification is provided indicating that relatively low-quality image data is obtained and will be used. Here, the process 400 can continue as normal without returning to a previous block.

In another example of an imaging system error (sometimes referred to as an “image data error”), control circuitry can be configured to detect (at/before/after block 418) that image data from an imaging system does not satisfy one or more criteria. In response, the control circuitry can provide a notification indicating that the image data does not meet certain requirements and/or to recapture image data with the imaging system (perform block 418 again). In some cases, the image data does not satisfy the one or more criteria when a directional tilt/angulation of the imaging system with respect to the patient body (e.g., cranial/caudal angle) is not within a predetermined range. As such, the notification can indicate that the directional tilt/angulation of the imaging system is not proper and/or to position the imaging system at a particular directional tilt/angulation. In response to receiving user input confirming that the imaging system is positioned properly, the control circuitry can return to block 418.

In a further example of an imaging system error (sometimes referred to as an “image data error”), control circuitry can be configured to detect (at/before/after block 418) that image data from an imaging system is missing data, such as one or more image slices, and/or that a transfer error occurred. In response, the control circuitry can provide a notification indicating to recapture image data with the imaging system (perform block 418 again). In response to receiving user input to restart image generation, the control circuitry can return to block 418.

FIG. 5 illustrates an example flow diagram of the process 500 for determining a spatial relationship between elements depicted within image data in accordance with one or more examples.

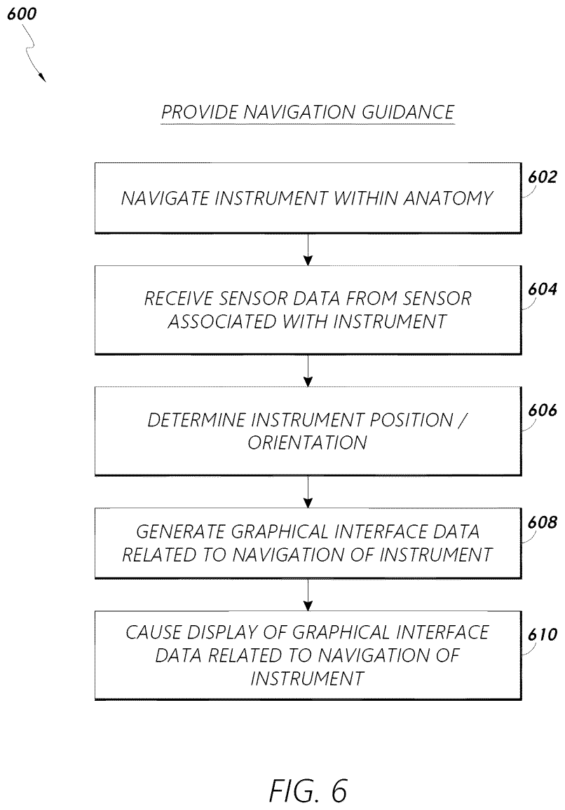

At block 502, the process 500 includes causing display of image data depicting internal anatomy. For example, control circuitry can cause display of image data received from or generated by an imaging system. In examples, causing display of the image data includes displaying the image data via a display/display screen. In other examples, causing display of the image data includes sending/providing the image data to a display/display device for display. In some cases, the image data can be displayed via an interface, such as the interface illustrated in FIG. 7 and/or 8 discussed in further detail below.

At block 504, the process 500 includes receiving user input identifying a target in the image data. For example, control circuitry can be configured to receive user input (also referred to as “user input data”) via an I/O component of a control/robotic system. The user input can include a selected/tagged location on the image data representing/identifying a target (e.g., a center of the target or another portion). In some cases where the image data includes multiple images/slices of image data (also referred to as “image slices”), the user can navigate through the slices of image data to identify the slice that depicts the target with the largest volume/area or other desired characteristics. The user can provide input to designate the center of the target in such image data. However, the user can designate the target in any image slice and/or any portion of a target. Each image slice can be a cross-sectional image. An example user interface and techniques for designating a target are discussed below in reference to FIG. 7.

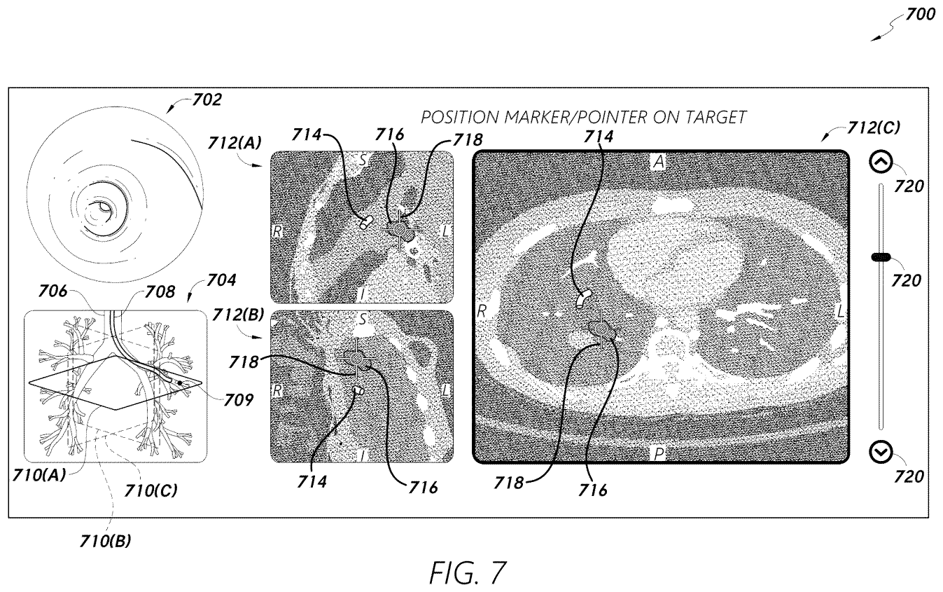

At block 506, the process 500 includes receiving user input identifying an instrument in the image data. For example, control circuitry can be configured to receive user input via an I/O component of a control/robotic system. The user input can include a selected/tagged location on the image data representing/identify an instrument (e.g., a distal end/tip or another portion of an endoscope, a distal end/tip or another portion of an instrument/tool deployed through the endoscope, a distal end/tip or portion of another instrument depicted in the image data, etc.). In some cases where the image data includes multiple images/slices of image data, the user can navigate through the slices of image data to identify the distal end of the instrument. The user can provide input to designate the distal end of the instrument in the image data. The user can designate any portion of the instrument in any image slice. An example user interface and techniques for designating an instrument are discussed below in reference to FIG. 8.

At block 508, the process 500 includes analyzing the image data to identify a position and/or orientation of an instrument (and/or target). For example, the user input received at block 506 on the image data can indicate a location/point of interest for an instrument. Here, the image data can be analyzed with one or more image processing techniques to detect/identify one or more image features that are within a predetermined proximity to the location/point of interest indicated in the user input. The one or more image features can be analyzed to identify certain image features that relate to an instrument, such as image features that include known/predetermined characteristics. An image feature can include an edge, corner, texture, intensity, etc. Once the instrument is identified in the image data, an orientation of the instrument can be determined, such as by identifying/mapping a longitudinal axis through a center of the distal end/portion of the instrument, wherein the orientation of the instrument can correspond to the orientation of the longitudinal axis. In some cases, a similar analysis can be performed to determine a position/orientation of a target, such as based on user input received at block 504.

Further, in examples, the image data can be analyzed without user input to estimate a location/point of interest in the image data, such as based on characteristics of an instrument. For instance, one or more image processing techniques can analyze the image data to detect/identify points/regions of interest that are associated with known/predetermined shapes, sizes, and/or other characteristics that are classified as being associated with an instrument. In some cases, a similar analysis can be performed to determine or estimate a position/orientation of a target. In examples, machine learning or other Artificial Intelligence (AI) techniques can be used to train an algorithm/model to detect/identify an instrument within image data. Such techniques can be trained based on training data that is manually or automatically tagged, such as image data that is tagged to designate an instrument therein. The algorithm/model can be used as part of one or more image processing techniques.

Although block 508 is shown as part of the process 500, in some cases block 508 is eliminated or performed for a certain element(s) within the image data. In one example, the process 500 can determine a location of an instrument/target based on a coordinate(s) associated with user input (e.g., coordinates of a user selection on image data). Here, image processing may or may not be performed.

At block 510, the process 500 includes causing display of an instrument indicator on the image data. For example, control circuitry can be configured to cause display of an instrument indicator representing a distal end of the instrument, wherein an orientation of the instrument indicator is aligned to the determined orientation of the instrument. The instrument indicator can be displayed in an overlaid manner on the image data to indicate a position and/or orientation of the distal end of the instrument. Although many examples discuss the instrument indicator as representing the distal end of the instrument, the instrument indicator can represent any portion of the instrument. In some cases, a target indicator representing the target is also displayed in a similar manner, such as on a determined location in the image data. As similarly noted above, causing display of the instrument/target indicator can include displaying the instrument/target indicator, sending/providing interface data to a display/display device for display, etc.

At block 512, the process 500 includes receiving user input including an adjustment and/or confirmation of the instrument indicator. For example, the instrument indicator can be configured to be manipulated based on user input, such as repositioned, reorientated, etc. In examples, control circuitry can receive user input to adjust the position/orientation of the instrument indicator on the image data, if needed, such that the instrument indicator covers the distal end of the instrument in the image data and/or is oriented to match the orientation of the distal end of the instrument in the image data. Once the instrument indicator is aligned to the satisfaction of the user, the user can provide user input including a confirmation that the instrument indicator is positioned accurately on the image data. In some case, a user can similarly provide user input regarding a target indicator that is displayed.

At block 514, the process 500 includes determining a spatial relationship between the instrument and the target based on the image data and/or the user input. In examples, control circuitry can be configured to determine the spatial relationship based on the user input that selects/identifies the instrument and/or target in the image data, based on image processing that identifies the instrument and/or target in the image data, and/or based on the location of the instrument/target indicator. As noted above, in some cases, the spatial relationship can include/represent a distance and/or vector between a distal end of the instrument and the target in the image data. For example, the spatial relationship can be represented with a vector between a point associated with the instrument indicator in a coordinate space and the point associated with the target in the coordinate space. Here, the vector can include a first set of coordinates for the start point and a second set of coordinates for the end point.

FIG. 6 illustrates an example flow diagram of the process 600 for providing navigation guidance for a medical instrument in accordance with one or more examples. The process 600 can be performed at various times. In one non-limiting illustration, the process 600 is performed as part of an intraoperative procedure where an instrument is navigated within anatomy to reach a target. However, the process 600 can be performed at other times.

At block 602, the process 600 includes navigating an instrument within the anatomy. For example, control circuity can be configured to cause the instrument to be controlled by a manipulator of a robotic system to navigate the instrument within the anatomy. In examples, a user can provide user input via a control system, causing the control system to generate robotic command data to control the robotic system to manipulate the instrument, such as to advance/retract, articulate, or otherwise move the instrument. The instrument can be navigated to reach a target (also referred to as a “anatomical site,” “procedure site,” or “desired anatomical site”), which can be associated with an object. Although discussed in the context of the robotic system manipulating the instrument, in some cases a user can manipulate the instrument partly or entirely by hand/manually.

At block 604, the process 600 includes receiving sensor data from a sensor associated with the instrument. For example, control circuitry can be configured to receive sensor data from a sensor associated with the instrument, such as while the instrument is navigated within the anatomy. The sensor can be positioned on a distal end of the instrument, along the instrument, and/or at other locations.

At block 606, the process 600 includes determining a position and/or orientation of the instrument. For example, control circuitry can be configured to use the sensor data to determine a position/orientation of the instrument. The position/orientation of the instrument can be a location of the instrument relative to an anatomical map, coordinate frame/space, etc. The position/orientation of the instrument can be represented with X, Y, Z coordinates, orientation information (e.g., roll/rotation, pitch, yaw), etc. In some instances, rotation/roll of the instrument (e.g., a clocking direction of a distal end of the instrument) is useful in cases where the instrument includes an off-axis imaging device, working channel, or other element. In examples, a pose of the instrument includes the position and orientation, which can be a pose of a distal end of the instrument.

In examples, at block 606, a direction in which the instrument is pointed/directed is also determined. For instance, an orientation of a distal end of the instrument can be determined, as well as a vector aligned with a longitudinal axis of an elongate shaft of the instrument and extending from the distal end thereof, such as to indicate a direction that the distal end of the instrument is pointed.

At block 608, the process 600 includes generating graphical interface data related to navigation of the instrument. For example, control circuitry can be configured to generate graphical interface data (sometimes referred to as “navigation data”) indicating the location of the instrument and/or location of a target. The graphical interface data can include/represent an anatomical map, wherein the location of the instrument/target can be depicted/represented relative to the anatomical map. As such, the graphical interface data can indicate the spatial relationship between the instrument and the target.

At block 610, the process 600 includes causing display of the graphical interface data within an interface. For example, control circuitry can cause the graphical interface data to be displayed via a display associated with a control system, robotic system, or other system. In examples, causing display of the graphical interface data includes displaying the graphical interface data via a display/display screen. In other examples, causing display of the graphical interface data includes sending/providing the graphical interface data to a display/display device for the display/display device to display the graphical interface data (e.g., content). The graphical interface data can include one or more interface elements (e.g., indicators, icons, visualizations, etc.) indicating the location/orientation of the instrument within the anatomy, the location/orientation of the target, anatomical features, etc. In examples, the graphical interface data includes an anatomical map, such as a virtual anatomical map or other map of the anatomy. In examples, causing display of graphical interface data or other types of interface data includes using the graphical interface data to generate displayable data.

In examples, one or more of blocks 604-610 are performed as the instrument is navigated within the anatomy. For example, the location of the instrument can be tracked and displayed in real-time to assist a user in navigating the instrument within the anatomy. Here, the user can view a real-time location of the instrument relative to an anatomical map and/or target, which can assist the user in reaching the target with the instrument.

Although various examples are discussed in the context of generating/providing navigation information in the form of displayable data (e.g., graphical interface data that is displayable), the techniques can similarly be performed to generate/provide other types of output data, such as in the form of haptic feedback, audio output, olfactory output, augmented reality output, light output (e.g., blinking lights), projected output, etc. Further, navigation data can be generated that may not be configured for display/output. For example, navigation data can include any information of the graphical interface data in a displayable or non-displayable format.

### Example Interfaces

FIGS. 7, 8, and 9 illustrate example interfaces 700, 800, and 900 (sometimes referred to as “graphical user interfaces (GUIs)”), respectively, to facilitate various functionality associated with updating navigation guidance in accordance with one or more examples.

FIGS. 7 and 8 illustrate the example interfaces 700 and 800 to provide intraoperative information regarding an instrument/target and/or to receive input regarding a location of a target/instrument within anatomy. The interface 700 of FIG. 7 (also referred to as “the target selection interface 700”) enables a user to select a location of the target within the image data, while the interface 800 of FIG. 8 (also referred to as “the instrument selection interface 800” enables the user to select a location of the instrument within the image data.

As shown, the interfaces 700 and 800 include an instrument view/section 702 providing image data captured from a distal end of the instrument (e.g., endoscope) within the anatomy. For example, the instrument view 702 can display real-time image data from a camera/imaging device located at a distal end of the instrument. Here, the real-time image data depicts internal anatomy, such as an anatomical lumen in which the instrument is located.

Further, the interfaces 700 and 800 include a map view/section 704 providing a model/representation of an anatomical map 706 (e.g., a virtual representation) and instrument/target location information. The map view 704 depicts the instrument within the anatomy with an instrument representation/indicator 708, which extends through the anatomical map 706 in this example. The map view 704 also depicts a location of a target with a target representation/indicator 709. In this example, the map view 704 depicts 3D information, such as the anatomical map 706 in 3D, and planes 710 associated with image views 712(A)-712(C). The user can select one of the image views 712 and the corresponding one of the planes 710 can be selected/marked, or vice versa, thereby assisting the user in referencing the image views 712. The planes 710 can be orthogonal to each other, as shown.

The image views 712 each include image data captured by an imaging system, such as a CT imaging system, X-Ray imaging system, etc. The image views 712 can each include image data representing a slice/layer of the internal anatomy, such as a 2D slice/layer. In examples, image data from the imaging system includes certain characteristics that are different than other forms of image data. For example, image data from the imaging system can represent/present different tissue types with different grayscale values. The image data can also include other features common to the type of imaging system used. In the examples of FIGS. 7 and 8, the image data is CT image data.

As shown in FIGS. 7 and 8, the image views 712 depict/show various anatomical features that can be identified by individuals, such as an individual trained to examine CT image data. For example, the image views 712 show an instrument feature 714 representing an instrument within the anatomy and a target feature 716 representing a target within the anatomy. Here, the instrument feature 714 (also referred to as “image feature 714”) represents a distal end of the instrument (e.g., a scope), while the target feature 716 (also referred to as “image feature 716”) represents a pulmonary nodule within the lungs. However, other features can be depicted/represented.

The interfaces 700 and 800 also include control/interface elements 720 configured to adjust one of the image views 712. For example, a user can move the selected image view 712(C) up and down (e.g., scroll) by using the control elements 720. This can allow the user to position the desired portion of the image within the center of the image view 712(C) or otherwise view features within the image view 712(C). Alternatively or additionally, the control/interface elements 720 can allow the user to navigate through multiple image slices associated with the anatomy.

As noted above, the interfaces 700 and 800 can enable a user to select locations of an instrument and/or target within the image views 712. For example, as shown in FIG. 7, the target selection interface 700 can request that the user position a marker/pointer on a target within the image data of one or more of the image views 712. The target can include certain characteristics that are recognizable by a trained individual. Here, the user moves an indicator 718 (also referred to as a “marker” or a “reticle”) within the target selection interface 700 to place the indicator 718 on the target feature 716 representing the target. The user can seek or be requested to place the indicator 718 on a center/central portion of the target feature 716, such that the indicator 718 in each of the image views 712 is placed on the center of the target feature 716 in the respective image view 712. Although the indicator 718 can be properly placed on the target feature 716 when the indicator 718 is located over at least a portion of the target feature 716. As shown, each of the image views 712 can include a respective indicator 718, wherein moving one of the indicators 718 may cause one or more of the other indicators 718 to move. As such, the indicators 718 can move in cooperation. In examples, the user may be requested to place the indicator 718 in each of the three views 712, wherein the user may need to provide input to adjust a position of the indicator in a respective image view 712 to ensure that the three indicators 718 are positioned properly (e.g., at the 3D center of the target).

Upon placing the indicator 718 as desired, the user can provide user input indicating that the location is confirmed/identified/tagged. The user input can be provided via the target selection interface 700 (e.g., selecting a confirm button) or otherwise. This can cause a location/orientation of the target to be determined, such as relative to a coordinate frame associated with the imaging system, a coordinate frame associated with image data captured by the imaging system, and/or another coordinate frame. In examples, the location/orientation of the target in an image coordinate frame can be converted to another coordinate frame, such as a sensor/EM coordinate frame used to tract the instrument (e.g., another intraoperative coordinate frame). In examples, the location of the target is represented with X, Y, Z coordinates.

Further, as shown in FIG. 8, the instrument selection interface 800 can request that the user position a marker/pointer on a distal end (or another portion) of an instrument within the image data of one or more of the image views 712. The distal end of the instrument can include certain characteristics that are recognizable by a trained individual. Here, the user moves the indicator 718 within the instrument selection interface 800 to place the indicator 718 on the instrument feature 714 representing the instrument within the anatomy. As noted above, each of the image views 712 can include a respective indicator 718, wherein moving one of the indicators 718 may cause one or more of the other indicators 718 to move. As such, in a similar fashion as that discussed above for the target, the user can place the indicator 718 in any of the image views 712 on the instrument feature 716. In examples, the user may be requested to accurately place the indicator 718 in each of the three views 712, wherein the user may need to provide input to adjust a position of the indicator in a respective image view 712 to ensure that the three indicators 718 are positioned properly (e.g., at the distal end of the instrument). In some examples, the indicator 718 from FIG. 7 to designate the location of the target is included within the interface 800 of FIG. 8 in addition to the indicator 718 in FIG. 8 to designate the instrument. This may allow the user to view the location of the target while attempting to locate the distal end of the instrument.

Upon placing the indicator 718 as desired, the user can provide user input indicating that the location is confirmed/identified/tagged. The user input can be provided via the instrument selection interface 800 (e.g., selecting a confirm button) or otherwise. This can cause a location/orientation of the instrument to be determined, such as relative to a coordinate frame associated with the imaging system, a coordinate frame associated with image data captured by the imaging system, and/or another coordinate frame. In examples, the location/orientation of the instrument in an image coordinate frame can be converted to another coordinate frame, such as a sensor/EM coordinate frame used to tract the instrument. In examples, the location/orientation of the instrument includes a location/orientation of a distal end of the instrument represented with X, Y, Z coordinates and/or roll/rotation, pitch, yaw.

In some examples of determining the orientation/position of the instrument, a message/notification can be presented via the instrument selection interface 800 or another interface requesting that the user rotate/move image data until the instrument faces the user (e.g., with a longitudinal axis of the instrument extending into and out of the display screen, such as perpendicular the display screen). For example, the user can provide input to update one of the image views 712 such that the instrument feature 714 is positioned to represent the instrument extending out of the display screen (e.g., the distal end of the instrument is pointed at the user). The user can provide input to indicate that the instrument is aligned as requested. This alignment can assist in analyzing the image data to determine the orientation/position of the instrument, in some cases. For instance, a longitudinal axis of the instrument can be determined as extending perpendicular to the display screen. However, in many examples, the instrument may not need to be aligned in such a manner to determine the orientation/position of the instrument.

In the illustrated examples of FIGS. 7 and 8, the indicator 718 takes the form of crosshairs/reticle; however, any type of indicator/user interface element can be used to mark, annotate, select, or otherwise designate a location of an element. To illustrate, the indicator 718 can take the form of a cursor, pointer, circle, or any other shape or form.

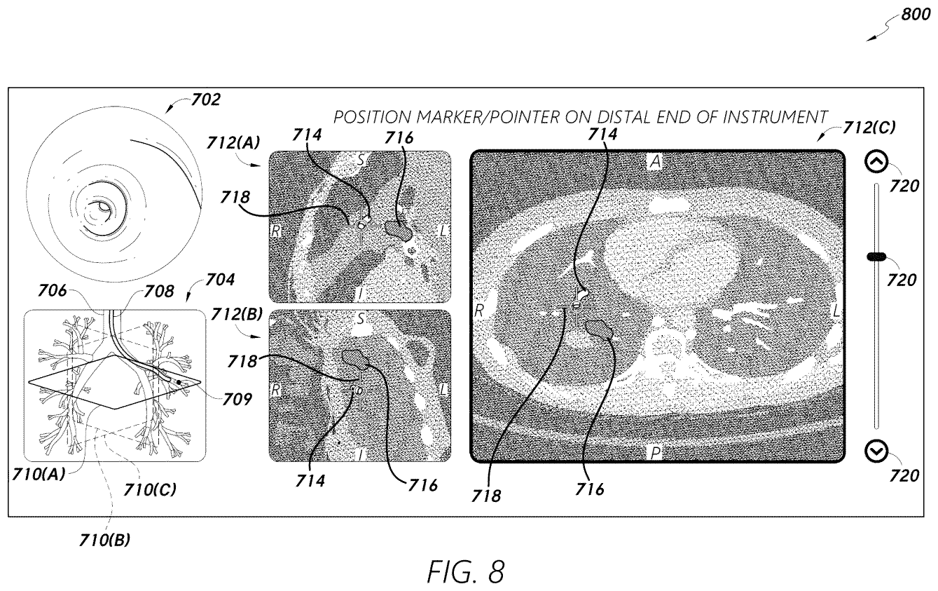

FIG. 9 illustrates the example interface 900 to provide input regarding a position and/or orientation of a distal end of an instrument (e.g., to adjust/confirm a position of the instrument). In examples, the interface 900 is provided upon the user identifying/tagging the location of the target and/or location of the instrument via the target selection interface 700 and/or instrument selection interface 800. For instance, based on user input that designates the location of the target and/or instrument within the interface 700/800, image data can be analyzed to determine or estimate an orientation/position of the instrument, as discussed herein. Information regarding the orientation/position of the instrument can then be displayed to the user via the interface 900 to confirm and/or adjust the orientation/position.

As shown, the interface 900 includes views/sections 902, 904, and 906 showing an instrument indicator 908 (also referred to as “tip indicator 908”) positioned on an instrument 910 depicted in image data (also referred to as “instrument feature 910” or “image feature 910”). Here, the view 902 illustrates a 3D view of image data captured by an imaging system (e.g., 3D CT image/model data), while the views 904 and 906 illustrate 2D views of the image data (e.g., 2D CT image data from different perspectives/planes). The instrument indicator 908 can initially be placed on the image at a location and/or in an orientation determined based on image processing and an initially selected user location (as discussed above in reference to FIG. 8). The instrument indicator 908 can be displayed in an overlaid manner on (e.g., on top of) image data.

In examples, the user can interact/manipulate the instrument indicator 908 to align the instrument indicator 908 with the instrument 910. For instance, the instrument indicator 908 can be a selectable/movable interface element, wherein the user can select and move the instrument indicator 908 to a desired location within any of the views 902-906. Further, in examples, the interface 900 can include interface controls/elements 912, 914 to reposition/align the instrument indicator 908 on the instrument 910. The interface controls 912 can cause the instrument indicator 908 (or the image data, in some cases) to move up, down, left, and right relative to the image data. Meanwhile, the interface control 914 can cause the instrument indicator 908 to rotate, thereby adjusting the orientation of the instrument indicator 908. The interface controls 912, 914 are illustrated for the view 904, since the view 904 is selected. However, the interface controls 912, 914 can similarly be provided for other ones of the views 902, 906 when the other view is selected. This can allow the user to reposition the instrument indicator 908 using the interface controls 912, 914 in any of the views 902-906.

In any event, the user can provide input to change a position and/or orientation of the instrument indicator 908, if desired, to ensure that the instrument indicator 908 is accurately positioned over the instrument 910 depicted in the image data. In examples, moving the instrument indicator 908 in one of the views 902-906 can cause the instrument indicator 908 to move in another one of the views 902-906. That is, movement of the instrument indicators 908 can be correlated for the different views 902-906.

As shown, the interface 900 can provide an image/indication 916 of an accurately positioned instrument indicator on a distal end of an instrument depicted and/or an image/indication 918 of inaccurately positioned instrument indicator. This can assist the user in aligning the instrument indicator 908 on the instrument 910 within the views 902-906. Further, in examples, such as that shown in FIG. 9, the interface 900 includes an instrument view/section 920 providing image data captured from a distal end of the instrument within the anatomy. Although the instrument view 920 may not be displayed in some examples.

Upon aligning the instrument indicator 908 as desired, the user can provide user input confirming the position/orientation of the instrument indicator 908. The user input can be provided via the interface 900 (e.g., selecting a confirm button) or otherwise. This can cause a location/orientation of the instrument to be determined, such as relative to a coordinate frame associated with the imaging system, a coordinate frame associated with image data captured by the imaging system, and/or another coordinate frame. As such, a location of the instrument indicator 908 can be translated/mapped to position within a coordinate frame. In examples, the location/orientation of the instrument is represented with X, Y, Z coordinates and/or roll/rotation, pitch, yaw.

In examples, upon confirming the position/orientation of the instrument indicator 908 and/or determining the location/orientation of the instrument, a spatial relationship between the target and the instrument can be determined. The spatial relationship can be represented with a vector from the user-provided instrument location to the user-provided target location. This spatial relationship can be used to update navigation information.

In some examples of aligning the instrument indicator 908 in the context of FIG. 9, the interface 900 can limit the degrees of freedom for placing/moving the instrument indicator 908. For instance, the interface 900 can provide two orthogonal views, such as the view 904 corresponding to a first plane and the view 906 corresponding to a second, orthogonal plane, wherein in each view the user can rotate/translate/move the instrument indicator 908 within the plane of that view. This can assist the user in accurately aligning the instrument indicator 908, such as by allowing the user to position the instrument indicator 908 in a particular view/plane at a time without having to consider the 3D location of the instrument indicator 908 all at once.

Although elements are displayed within a particular interface in FIGS. 7-9, any of the elements can be eliminated and/or combined in different formats. For example, a view that is illustrated in one interface can be eliminated and/or provided in another interface.

### Example Navigation Update

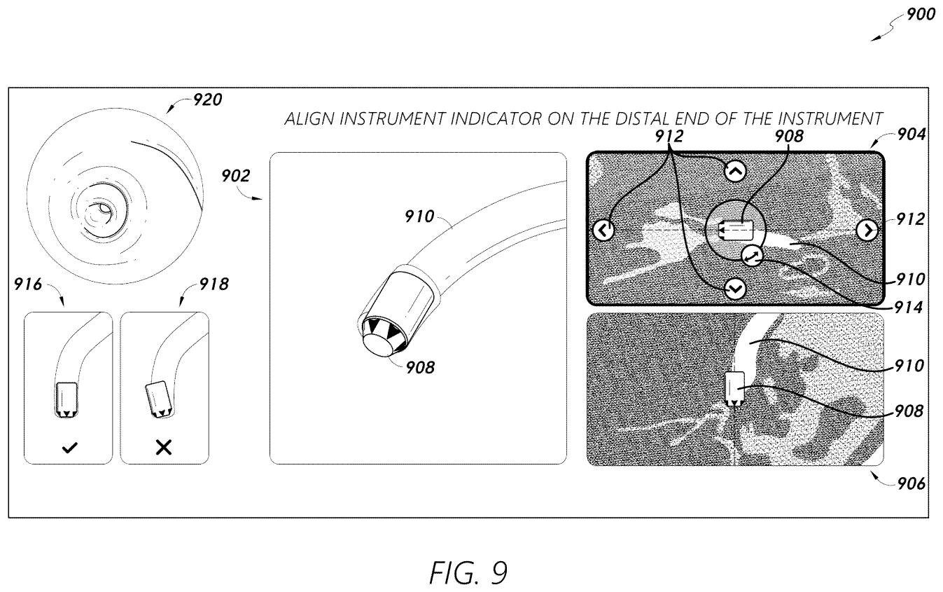

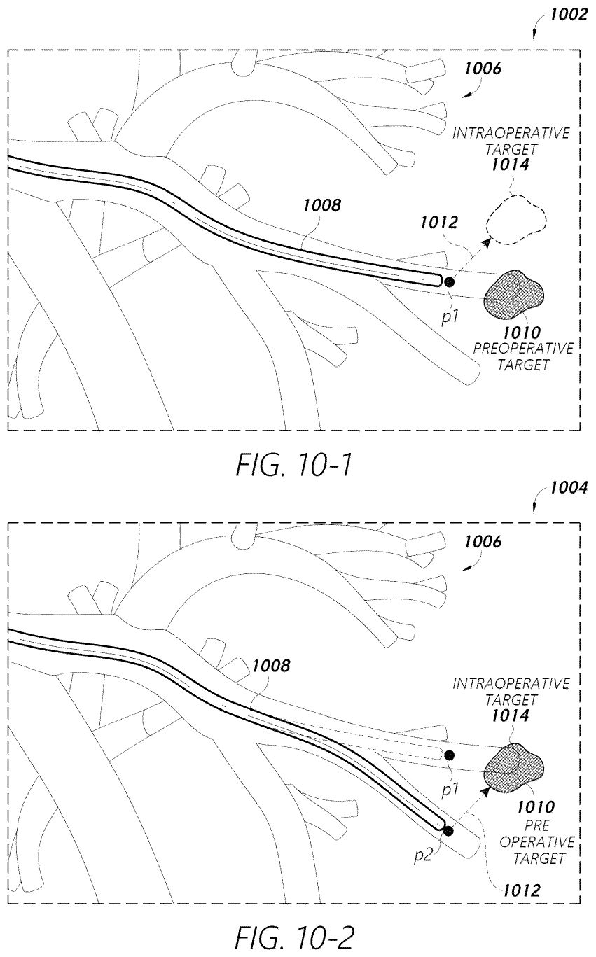

FIGS. 10-1 and 10-2 illustrate an example of updating navigation information/data related to a location of an instrument in accordance with one or more examples. FIG. 10-1 illustrates a representation 1002 of initial navigation information indicating an assumed/estimated location of an instrument, while FIG. 10-2 illustrates a representation 1004 of updated navigation information indicating the actual location of the instrument. The navigation information can be displayable or non-displayable data. In FIGS. 10-1 and 10-2, the navigation information is provided with various representations to illustrate aspects of the navigation information. However, such information may or may not be displayed to a user.

As shown in FIG. 10-1, the navigation information can include an anatomical map 1006 representing anatomical lumens/pathways and/or other features within the anatomy. In this example, the anatomical map 1006 represents a portion of the bronchial tree/bronchial network of the lungs of a patient. In examples, the anatomical map 1006 is a preoperative map that is registered/aligned to an intraoperative coordinate frame/space. The navigation information also can include/indicate a location of an instrument 1008 within the anatomical map 1006. Further, the navigation information can initially include/indicate a location of a preoperative target 1010 within the anatomical map 1006. Here, a distal end of the instrument 1008 is positioned at a location p1, wherein the preoperative target 1010 is axially aligned with the instrument 1008 and located relatively close to the instrument 1008.

FIG. 10-2 illustrates the navigation information after the navigation information has been updated based on the techniques discussed herein. That is, the updated navigation information indicates the actual, more accurate intraoperative location of the instrument 1008. As shown in FIGS. 10-1 and 10-2, a vector 1012 can represent a spatial relationship of the distal end of the instrument 1008 to the intraoperative target 1014, wherein the vector 1012 can be determined based on techniques discussed herein, such as a user designating a location of a distal end of the instrument and a location of the target based on intraoperative image data. Although the location of the instrument 1008 is updated in this example, the anatomical map 1006 and/or other information can be updated, as discussed herein.

FIG. 10-1 illustrates that the instrument 1008 is not actually positioned accurately relative to the target. Thus, the navigation information is updated to reposition the instrument 1008 at the accurate position relative to the target. This can be accomplished by shifting/moving a location of the distal end of the instrument 1008 to point p2 within the anatomical map 1006 and aligning the intraoperative target 1014 with the preoperative target 1010, as shown in FIG. 10-2. As such, the target is now accurately positioned up and to the right relative to the view of FIG. 10-2 and not right in front of the instrument 1008 as previously indicated in FIG. 10-1.

### Example User Interface Update

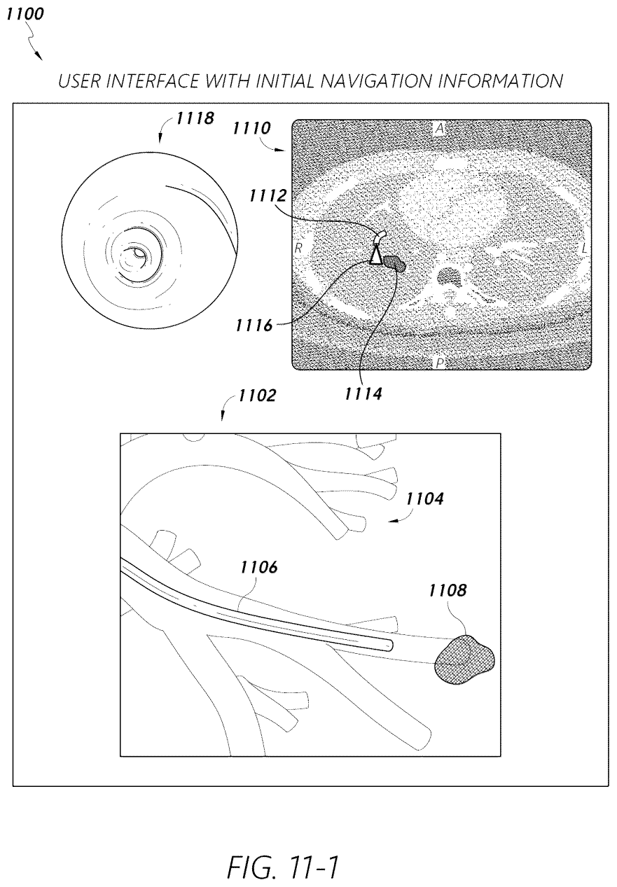

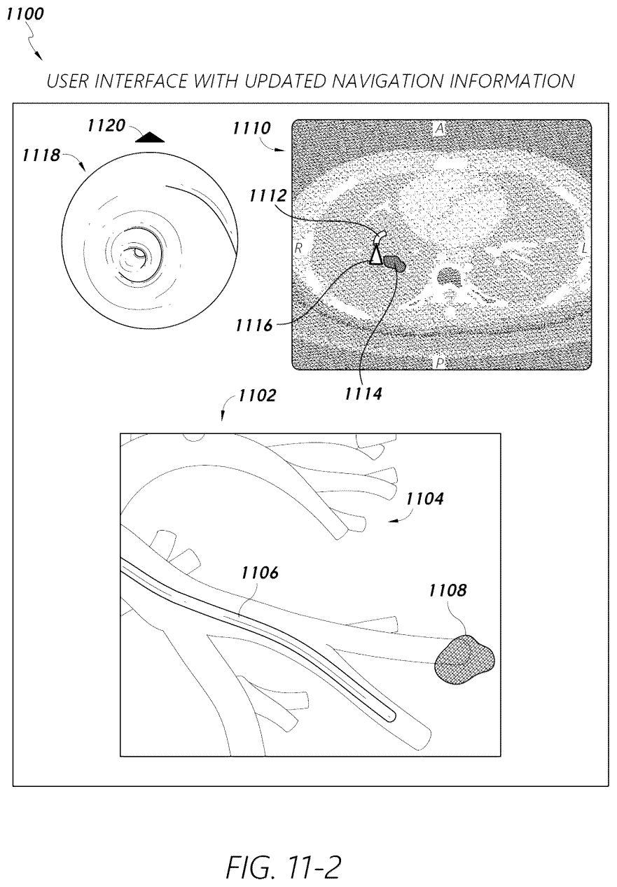

FIGS. 11-1 and 11-2 illustrate an example interface 1100 (also referred to as “user interface 1100”) that provides/presents graphical interface data regarding navigation information of an instrument before and after updating the navigation information in accordance with one or more examples. FIG. 11-1 illustrates initial navigation information, while FIG. 11-2 illustrates updated navigation information. The interface 1100 can be displayed via a display/display device.

In examples, the interface 1100 is provided during a navigation update. For example, a navigation interface (not shown) can be provided during normal navigation of an instrument, which can provide various views to assist a user in navigating the instrument within the anatomy. The navigation interface can include one or more of the views of the interface 1100 or other views. In any event, in some examples, upon initializing a navigation update, the interface 1100 of FIG. 11-1 can initially be displayed. Then, after updating navigation information, the interface 1100 of FIG. 11-2 can be displayed. However, the interface 1100 of FIGS. 11-1 and/or 11-2 can be provided at other times.

As shown in FIGS. 11-1 and 11-2, the interface 1100 includes a map view/section 1102 providing a model/representation of an anatomical map 1104 (e.g., a virtual map. The anatomical map 1104 can be a 2D or 3D representation. The map view 1102 shows an instrument indicator 1106 representing the instrument within the anatomy. The map view 1102 also shows a target indicator 1108 representing the target within the anatomy. In examples, the anatomical map 1104 and/or target are based on preoperative data and registered/aligned to an intraoperative coordinate frame/space.

In examples, the interface 1100 also includes an image view 1110 including image data captured intraoperatively (or preoperatively, in some cases). For example, the image view 1110 can display CT image data captured by a CT imaging system; however, other forms of image data can be provided. In this example, the image data depicts an instrument 1112 and a target 1114, which are outlined for ease of illustration. The image view 1110 can also provide a direction indicator 1116 indicating a direction that the instrument 1112 is pointing. The direction indicator 1116 can be oriented based on an orientation/position of the instrument determined by any of the techniques discussed herein.

In examples, the interface 1100 also includes an instrument view/section 1118 providing image data captured from a distal end of the instrument within the anatomy. Although the interface 1100 includes three non-limiting views 1102, 1110, and 1118 for illustration purposes, any of the views 1102, 1110, and 1118 and/or elements of the views 1102, 1110, and 1118 can be eliminated and/or formatted/combined in other manners.

As shown in FIG. 11-1, the map view 1102 of the interface 1100 initially includes the instrument indicator 1106 positioned in a first manner/location within the anatomical map 1104, such as within a first anatomical lumen. Here, the instrument indicator 1106 shows that the instrument is positioned in line with the target indicator 1108. Whereas FIG. 11-2 shows the interface 1100 after a navigation update, such as by using one or more of the techniques discussed herein. In FIG. 11-2, the map view 1102 shows the instrument indicator 1106 positioned in a different manner/location within the anatomical map 1104, such as a second anatomical lumen. The updated interface 1100 of FIG. 11-2 now indicates a more accurate position of the instrument relative to the target and/or the anatomical map 1104.

In this example, the interface 1100 is updated by maintaining most elements of the map view 1102 in a relatively fixed position. In particular, the anatomical map 1104 and target indicator 1108 are maintained/displayed at fixed positions within the interface 1100/map view 1102. Meanwhile, the instrument indicator 1106 is moved/shifted/updated to reflect the updated navigation information. In examples, a shaft/elongate member of the instrument indicator 1106 can be maintained in the same form while the instrument indicator 1106 is shifted. That is, the shaft of the instrument indicator 1106 can maintain the same shape/curve during the update (e.g., include the same shape/form in FIGS. 11-1 and 11-2). In other examples, the shaft of the instrument indicator 1106 is updated, such as to show a slightly different curve/shape.

Although the example of FIGS. 11-1 and 11-2 updates the instrument indicator 1106 while maintaining other elements in the map view 1102, in other examples the instrument indicator 1106 is maintained in a fixed position and the other elements are updated. For instance, the instrument indicator 1106 can maintain a fixed position within the map view 1102 during the transition of the interface 1100 from FIG. 11-1 to FIG. 11-2, while the anatomical map 1104 and/or target indicator 1108 can be shifted/moved.

In some examples, the instrument view 1118 includes an indicator 1120 indicating a direction of the target relative to the instrument view 1118, as shown in FIG. 11-2. The indicator 1120 can indicate a location of the target relative to a distal end of the instrument, which can be based on a coordinate frame of the distal end of the instrument. Here, the indicator 1120 shows that the target is not axially aligned with the instrument, as initially understood based on the map view 1102 in FIG. 11-1. In particular, the indicator 1120 shows that the target is upwards relative to the view 1118 of FIG. 11-2.

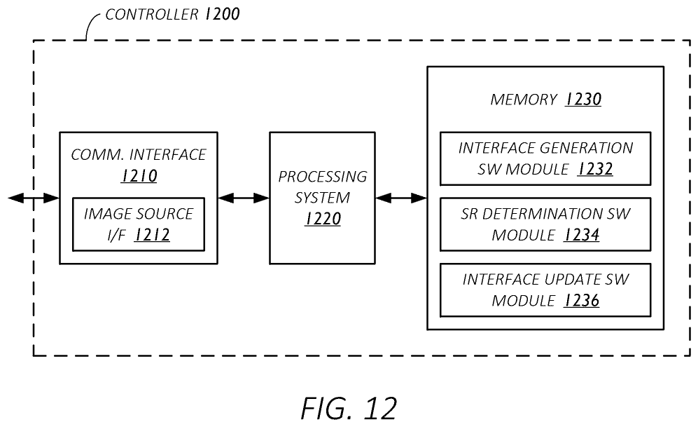

FIG. 12 shows another block diagram of an example controller 1200 for a medical system, according to some implementations. In some implementations, the controller 1200 may be one example of the control circuitry 208 and/or 224 of FIG. 2. More specifically, the controller 1200 is configured to guide navigation of an instrument within an anatomy.

The controller 1200 includes a communication interface 1210, a processing system 1220, and a memory 1230. The communication interface 1210 is configured to communicate with one or more components of the medical system. More specifically, the communication interface 1210 includes an image source interface (I/F) 1212 for communicating with one or more image sources (such as the CT imaging system 310 and/or the fluoroscopy imaging system 312 of FIG. 3). In some implementations, the image source I/F 1212 may receive image data depicting the anatomy having the instrument disposed therein, where the image data is captured by an imaging system positioned external to the anatomy.

The memory 1230 may include a non-transitory computer-readable medium (including one or more nonvolatile memory elements, such as EPROM, EEPROM, Flash memory, or a hard drive, among other examples) that may store the following software (SW) modules: an interface generation SW module 1232 to generate a graphical interface depicting a first spatial relationship between an instrument and a target within an anatomy; a spatial relationship (SR) determination SW module 1234 to determine a second spatial relationship between the instrument and the target within the anatomy based at least in part on the image data; and an interface update SW module 1236 to update the graphical interface to depict the second spatial relationship between the instrument and the target.

The processing system 1220 may include any suitable one or more processors capable of executing scripts or instructions of one or more software programs stored in the controller 1200 (such as in the memory 1230). For example, the processing system 1220 may execute the interface generation SW module 1232 to generate a graphical interface depicting a first spatial relationship between an instrument and a target within an anatomy. The processing system 1220 also may execute the SR determination SW module 1234 to determine a second spatial relationship between the instrument and the target within the anatomy based at least in part on the image data. The processing system 1220 may further execute the interface update SW module 1236 to update the graphical interface to depict the second spatial relationship between the instrument and the target.

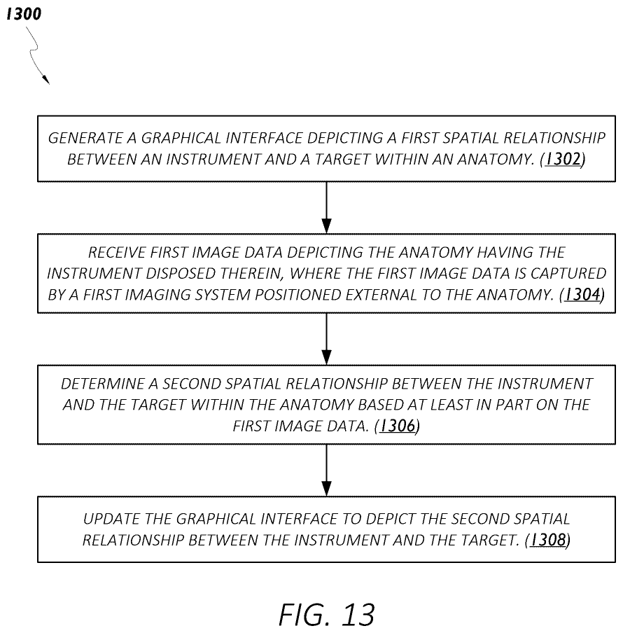

FIG. 13 shows an illustrative flowchart depicting an example operation 1300 for providing navigation guidance for an instrument within an anatomy, according to some implementations. In some implementations, the example operation 1300 may be performed by a controller for a medical system such as the controller 1200 of FIG. 12.

The controller generates a graphical interface depicting a first spatial relationship between an instrument and a target within an anatomy (1302). The controller also receives first image data depicting the anatomy having the instrument disposed therein, where the first image data is captured by a first imaging system positioned external to the anatomy (1304). The controller determines a second spatial relationship between the instrument and the target within the anatomy based at least in part on the first image data (1306). The controller further updates the graphical interface to depict the second spatial relationship between the instrument and the target (1308).

In some implementations, the determining of the second spatial relationship may include displaying one or more images of the anatomy based on the first image data, and receiving user input indicating at least one of a position the target or a position of the instrument in the one or more images. In some other implementations, the determining of the second spatial relationship may include estimating at least one of a position of the target or a position of the instrument based on the first image data.

In some aspects, the generating of the graphical interface may include receiving second image data depicting a position of the target within the anatomy, receiving sensor data from a sensor associated with the instrument, where the sensor data indicates a position of the instrument within the anatomy, and determining the first spatial relationship based on the second image data and the sensor data. In some implementations, the second image data may be captured by a second imaging system while the instrument is not disposed within the anatomy.

In some aspects, the determining of the second spatial relationship may include displaying an instrument indicator overlaid on one or more images depicting the anatomy with the instrument disposed therein based on the first image data and receiving user input for aligning the instrument indicator with a distal end of the instrument in the one or more images. In some implementations, the controller may further estimate a pose of the instrument based on the first image data, where the instrument indicator is overlaid on the one or more images based on the estimated pose of the instrument. In some implementations, the user input may include at least one of an adjustment to a position or orientation of the instrument indicator relative to the one or more images or an indication that the instrument indicator is aligned with the distal end of the instrument.

### Additional Implementation Description and Terminology

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

In the above description, various features are sometimes grouped together in a single example, figure, or description thereof for the purpose of streamlining the disclosure and aiding in the understanding of one or more of the various aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the subject matter herein disclosed and claimed below should not be limited by the particular examples described above, but should be determined only by a fair reading of the claims that follow.

In examples, certain ordinal terms (e.g., “first” or “second”) can be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example examples belong. It is further understood that terms, such as those defined in commonly used dictionaries, should be interpreted as having a meaning that is consistent with their meaning in the context of the relevant art and not be interpreted in an idealized or overly formal sense unless expressly so defined herein. As used herein, the term “patient” may generally refer to humans, anatomical models, simulators, cadavers, and other living or non-living objects.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A medical system comprising:
a first imaging system; and
control circuitry configured to:
generate a graphical interface depicting a first spatial relationship between an instrument and a target within the anatomy;
receive first image data depicting the anatomy having the instrument disposed therein, the first image data captured by the first imaging system while positioned external to the anatomy;
determine a second spatial relationship between the instrument and the target within the anatomy based at least in part on the first image data; and
update the graphical interface to depict the second spatial relationship between the instrument and the target.

2. The medical system of claim 1, wherein the first imaging system is a cone beam computed tomography (CBCT) imaging system.

3. The medical system of claim 1, wherein the determining of the second spatial relationship comprises:
displaying one or more images of the anatomy based on the first image data; and
receiving user input indicating at least one of a position of the target or a position of the instrument in the one or more images.

4. The medical system of claim 1, wherein the determining of the second spatial relationship comprises:
estimating at least one of a position of the target or a position of the instrument based on the first image data.

5. The medical system of claim 1, wherein the generating of the graphical interface comprises:
receiving second image data depicting a position of the target within the anatomy;
receiving sensor data from a sensor associated with the instrument, the sensor data indicating a position of the instrument within the anatomy; and
determining the first spatial relationship based on the second image data and the sensor data.

6. The medical system of claim 5, wherein the second image data is captured by a second imaging system while the instrument is not disposed within the anatomy.

7. The medical system of claim 1, wherein the determining of the second spatial relationship comprises:
displaying an instrument indicator overlaid on one or more images depicting the anatomy with the instrument disposed therein based on the first image data; and
receiving user input for aligning the instrument indicator with a distal end of the instrument in the one or more images.

8. The medical system of claim 7, wherein the control circuitry is further configured to:
estimate a pose of the instrument based on the first image data, the instrument indicator overlaid on the one or more images based on the estimated pose of the instrument.

9. The medical system of claim 7, wherein the user input includes an adjustment to a position or orientation of the instrument indicator relative to the one or more images.

10. The medical system of claim 7, wherein the user input includes an indication the instrument indicator is aligned with the distal end of the instrument.

11. The medical system of claim 1, wherein the first spatial relationship is represented by a depiction of the instrument in a first pose relative to a position of the target and the second spatial relationship is represented by a depiction of the instrument in a second pose relative to the position of the target.

12. A method performed by a controller for a medical system, comprising:
generating a graphical interface depicting a first spatial relationship between an instrument and a target within an anatomy;
receiving first image data depicting the anatomy having the instrument disposed therein, the first image data captured by a first imaging system positioned external to the anatomy;
determining a second spatial relationship between the instrument and the target within the anatomy based at least in part on the first image data; and
updating the graphical interface to depict the second spatial relationship between the instrument and the target.

13. The method of claim 12, wherein the determining of the second spatial relationship comprises:
displaying one or more images of the anatomy based on the first image data; and
receiving user input indicating at least one of a position the target or a position of the instrument in the one or more images.

14. The method of claim 12, wherein the determining of the second spatial relationship comprises:
estimating at least one of a position of the target or a position of the instrument based on the first image data.

15. The method of claim 12, wherein the generating of the graphical interface comprises:
receiving second image data depicting a position of the target within the anatomy;
receiving sensor data from a sensor associated with the instrument, the sensor data indicating a position of the instrument within the anatomy; and
determining the first spatial relationship based on the second image data and the sensor data.

16. The method of claim 15, wherein the second image data is captured by a second imaging system while the instrument is not disposed within the anatomy.

17. The method of claim 12, wherein the determining of the second spatial relationship comprises:
displaying an instrument indicator overlaid on one or more images depicting the anatomy with the instrument disposed therein based on the first image data; and
receiving user input for aligning the instrument indicator with a distal end of the instrument in the one or more images.

18. The method of claim 17, further comprising:
estimating a pose of the instrument based on the first image data, the instrument indicator overlaid on the one or more images based on the estimated pose of the instrument.

19. The method of claim 17, wherein the user input includes at least one of an adjustment to a position or orientation of the instrument indicator relative to the one or more images or an indication that the instrument indicator is aligned with the distal end of the instrument.

20. A controller for a medical system, comprising:
a processing system; and
a memory storing instructions that, when executed by the processing system, cause the controller to:
generate a graphical interface depicting a first spatial relationship between an instrument and a target within an anatomy;
receive image data depicting the anatomy having the instrument disposed therein, the image data captured by an imaging system positioned external to the anatomy;
determine a second spatial relationship between the instrument and the target within the anatomy based at least in part on the image data; and
update the graphical interface to depict the second spatial relationship between the instrument and the target.
